(19) United States
(12) Patent Application Publication
(10) Pub. No.: US 2023/0065522 A1
(43) Pub. Date: Mar. 2, 2023

(54) METHODS OF CANCER DETECTION USING PARPI-FL

(71) Applicant: Memorial Sloan Kettering Cancer Center, New York, NY (US)

(72) Inventors: Susanne KOSSATZ, New York, NY (US); Thomas REINER, New York, NY (US); Wolfgang WEBER, New York, NY (US); Snehal G. PATEL, New York, NY (US)

(21) Appl. No.: 17/846,690

(22) Filed: Jun. 22, 2022

Related U.S. Application Data
(63) Continuation of application No. 16/712,919, filed on Dec. 12, 2019, now abandoned.
Application No. 16/712,919 is a continuation of application No. 15/565,369, filed on Oct. 9, 2017, now abandoned.
(60) Provisional application No. 62/145,873, filed on Apr. 10, 2015.
(60) Provisional application No. 62/291,463, filed on Feb. 4, 2016.

(51) Int. Cl. A61K 49/00; A61B 5/00; A61B 5/055; A61K 9/00

(52) U.S. Cl. CPC A61K 49/0052; A61K 49/0073; A61K 49/0071; A61K 49/0021; A61B 5/0036; A61B 5/055; A61K 9/0014

(57) ABSTRACT

The present disclosure describes methods of use of a composition comprising PARPi-fl to be administered to the oral cavity (e.g., via topical application to surfaces of the oral cavity) followed by imaging of the oral cavity for detection of squamous cell carcinoma of the oral cavity (e.g., in vivo, e.g., in a dental office setting or intraoperatively). The results disclosed herein show that topically applied PARPi-fl and subsequent intraoperative imaging of oral cavities can improve surgical removal of squamous cell carcinoma cells compared to healthy cells.

### CROSS-REFERENCE TO RELATED APPLICATIONS

This application is a continuation of U.S. patent application Ser. No. 15/565,369, filed Oct. 9, 2017, which is a National Stage Application of PCT/US2016/026717, filed Apr. 8, 2016, which claims the benefit of U.S. Application Ser. No. 62/145,873 filed on Apr. 10, 2015 and U.S. Application Ser. No. 62/291,463 filed on Feb. 4, 2016, the disclosures of each of which are hereby incorporated by reference in their entireties.

### GOVERNMENT FUNDING

This invention was made with government support under Grant No. K25 EB016673-01 awarded by the NIH. The government has certain rights in this invention.

### FIELD OF THE INVENTION

This invention relates generally to the use of imaging probes for the diagnosis and treatment of cancer. In particular embodiments, the invention relates to a method for the non-invasive screening of a subject for oral squamous cell carcinoma (OSCC).

### BACKGROUND

Poly(ADP-ribose)polymerase 1 (PARP1), an enzyme that is activated to repair damaged DNA in the cells, is increased in cancer cells due to the higher genomic instability. For certain subtypes of cancer, inhibition of PARP1 and the associated perturbation of the DNA repair pathway have been shown to be a successful chemotherapeutic treatment regime, both in preclinical as well as clinical research settings. Because PARP1 is overexpressed in various forms of cancer, it has become a high-value target for the treatment, diagnosis, and staging of cancer. It appears that no one has demonstrated highly elevated PARP1 expression in human oral cancer specimens before.

Oral cancer is defined as a malignant neoplasm on the lip or in the mouth, affecting more than 40,000 patients in the United States in 2014. Of all malignant oral cancers, oral squamous cell carcinoma (OSCC) is by far the most common epithelial malignancy in the oral cavity, accounting for over 90% of all cases. While the disease is not as threatening as other types of cancer when detected early (83% 5-year survival for local disease), nearly half of all patients display distant metastases at the time of diagnosis due to the lack of accurate screening protocols and screening tools for this type of disease.

Currently, visual inspection of suspicious lesions is the most common way of diagnosing OSCC. Besides tissue sampling, different optical imaging techniques can be used to non-invasively survey the mucosal tissue (optical or fluorescent imaging) and to obtain a more accurate picture of oral cancer growth, its location, spread and number of diseased lesions. Optical methods like chemiluminescence, which examines the higher density of nuclei in malignant tissues; tissue fluorescence, which measures the higher autofluorescence of cancerous lesions due to higher chromatin/metabolite content and stromal/collagen changes; or the imaging of toluidine blue, a dark-blue stain that binds to the DNA of cells, that accumulates to a higher degree in malignant tissues; have been used to non-invasively determine the presence of oral cancer. However, these tools do not target a specific biomarker and lack specificity, resulting in either high false-positive or false negative rates. Low specificity particularly hampers the detection of small or precancerous lesions, where accurate detection would have the highest impact.

The most abundant radiotracer used in the clinic today is ¹⁸F-FDG, a glucose analog with high uptake in most types of cancer. However, the use of ¹⁸F-FDG requires significant infrastructure (e.g. tomography scanners, availability of the short-lived ¹⁸F radioisotope, close proximity of a medical cyclotron, specialized personnel, etc.). Furthermore, the administration of radioisotopes is always linked to radioactivity absorbed by both patient and healthcare professionals. Thus, radiolabeled imaging agents are not suitable for screening purposes, and only patients with suspected or confirmed disease are typically subjected to PET scans. In the case of oral cancer, a screening-tool for early detection is needed to improve patient outcome. Furthermore, a bimodal imaging agent for dual use in screening and PET scanning would be optimal to improve outcomes of oral cancer.

There is a need for a technology that can detect cancer cell populations and treat tissues that are still viable from one or more successfully ablated tumor(s). Moreover, there is a clinical need to develop a robust and reliable imaging agent for oral cancer in the oral cavity. This technology must be able to detect malignant growth of the mucosa while still local and treatable.

### SUMMARY

Presented herein is a method for the non-invasive screening of a subject for oral squamous cell carcinoma (OSCC). Methods described herein use poly(ADP-ribose)polymerase 1 (PARP1), a targeted small molecule imaging agent, as a diagnostic tool to identify oral squamous cell carcinoma (OSCC) and improve surgical removal of tumors by intraoperative imaging. PARPi-fl can be used to detect malignant growth in the oral cavity, e.g., in a dentist's office setup, using a macroscopic fluorescence scanning imaging device after topical application of PARPi-fl, which preferentially accumulates in areas of elevated PARP1 expression. Furthermore, the employment of a microscopic device, such as a fluorescence endoscope or a handheld confocal microscope can improve identification of tumor margins and residual tumor tissue during tumor removal surgery.

In one aspect, the invention is directed to a method for the non-invasive screening of a subject for oral squamous cell carcinoma (OSCC), the method comprising the steps of: administering a composition comprising PARPi-fl onto and/or into tissue in an oral cavity of the subject, wherein fl comprises a fluorophore; flushing the tissue in the oral cavity of the subject to reduce or remove unbound components of the composition while leaving bound components of the composition; and detecting fluorescence from the fluorophore after the administering and the flushing steps, thereby identifying potential OSCC.

In certain embodiments, the subject is a human patient.

In certain embodiments, the method takes place in a dentist office or other non-surgical setting.

In certain embodiments, the administering is topically administered.

In certain embodiments, the composition is a liquid composition.

In certain embodiments, the liquid composition is selected from the group consisting of an oral rinse, a mouthwash, a spray, an intravenous injection, and a local intramuscular injection.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the flushing comprises waiting a period of time following the administering step such that the tissue clears unbound components of the composition (e.g., via the lymphatic system, endocytosis).

In certain embodiments, the flushing comprises rinsing (e.g., with water or saline) and/or gargling (e.g., with water or saline).

In certain embodiments, potential OSCC is identified at the area of accumulation. In certain embodiments, potential OSCC is identified using a fluorescence scanning imaging device. In certain embodiments, potential OSCC is identified following exposure of the oral cavity tissue to excitation light.

In another aspect, the invention is directed to a method for intraoperative detection of a tumor margin and/or residual tumor tissue during tumor removal surgery, the method comprising the steps of: administering a composition comprising PARPi-fl onto and/or into a viewable tissue surface of the subject, wherein fl comprises a fluorophore; flushing the viewable tissue surface to reduce or remove unbound components of the composition while leaving bound components of the composition; and detecting fluorescence from the fluorophore after administration to the viewable tissue surface, thereby identifying the tumor margin and/or residual tumor tissue.

In certain embodiments, the tissue is from cancers of the aerodigestive tract, gastrointestinal tract, urinary tract, ovarian cancer, oral cancer, colorectal cancer, stomach cancer, bladder cancer, cervical cancer, retinal cancer, skin cancer, lung cancer, bronchus cancer, esophageal cancer, or any cancer that can be observed close to the tissue surface with a laparoscopic microscope (e.g., pancreatic, liver, kidney, spleen) or any cancer that is surgically resected, and where tissue margins can be observed (e.g., brain).

In certain embodiments, the administering is topically administered or intravenously administered.

In certain embodiments, the viewable tissue surface is viewable to a surgeon.

In certain embodiments, the viewable tissue surface is viewable by direct exposure or by a camera with access to the tissue surface (e.g., via endoscope).

In certain embodiments, the composition is a liquid composition. In certain embodiments, the liquid composition comprises a rinse.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety has a molecular weight no greater than about 500 Da, e.g., no greater than about 400 Da, e.g., no greater than about 300 Da.

In certain embodiments, the flushing comprises waiting a period of time following the administering step such that the tissue clears unbound components of the composition (e.g., via the lymphatic system, endocytosis).

In certain embodiments, the flushing comprises rinsing (e.g., with water or saline) and/or gargling (e.g., with water or saline).

In certain embodiments, the tumor margin is identified using a fluorescence scanning imaging device.

In certain embodiments, the tumor margin is identified following exposure of the viewable tissue surface to excitation light.

In another aspect, the invention is directed to a composition comprising: a PARP inhibitor conjugated to a fluorophore.

In certain embodiments, the PARP inhibitor is selected from the group consisting of AZD2281, AG014699 (rucaparib), ABT888 (veliparib), BSI201 (iniparib), BSI101, DR2313, FR 247304, GPI15427, GPI16539, M 4827, NU1025, NU1064, NU1085, PD128763, PARP Inhibitor H (INH2BP), PARP Inhibitor m (DPQ), PARP Inhibitor VHI (PJ34), PARP Inhibitor IX (EB-47), and TIQ-A.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the composition is a liquid composition.

In certain embodiments, the liquid composition is selected from the group consisting of an oral rinse, a mouthwash, a spray, an intravenous injection, and a local intramuscular injection.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In another aspect, the invention is directed to a method of assessing efficacy of a cancer therapy in a subject receiving treatment for an oral carcinoma, the method comprising administering to the subject the composition.

In certain embodiments, the cancer therapy comprises chemotherapy, radiation, or surgery.

In certain embodiments, the administering occurs subsequent to the cancer therapy.

In certain embodiments, the method further comprises administering a composition comprising ¹⁸F-PARPi to the subject.

In certain embodiments, the composition is in the same or in a different composition than the composition comprising PARPi-fl.

In certain embodiments, the administered composition enables PET imaging.

In certain embodiments, two orthogonal imaging modalities, PET for ¹⁸F-PARPi and optical imaging for PARPi-fl are conducted, thereby enabling screening (e.g., via optical imaging) and staging (e.g., via PET imaging) of disease.

In another aspect, the invention is directed to a composition comprising PARPi-fl, wherein fl comprises a fluorophore, for use in a method of in vivo diagnosis of oral squamous cell carcinoma (OSCC) in a subject, wherein the in vivo diagnosis comprises: delivering the composition onto and/or into tissue in an oral cavity of the subject; flushing the tissue in the oral cavity of the subject to reduce or remove unbound components of the composition while leaving bound components of the composition; and detecting fluorescence from the fluorophore after the administering and the flushing steps, thereby identifying potential OSCC.

In certain embodiments, the administering is topically administered.

In certain embodiments, the composition is a liquid composition. In certain embodiments, the liquid composition is selected from the group consisting of an oral rinse, a mouthwash, a spray, an intravenous injection, and a local intramuscular injection.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the flushing comprises waiting a period of time following the administering step such that the tissue clears unbound components of the composition (e.g., via the lymphatic system, endocytosis).

In certain embodiments, the flushing comprises rinsing (e.g., with water or saline) and/or gargling (e.g., with water or saline).

In certain embodiments, potential OSCC is identified at the area of accumulation. In certain embodiments, potential OSCC is identified using a fluorescence scanning imaging device. In certain embodiments, potential OSCC is identified following exposure of the oral cavity tissue to excitation light.

In another aspect, the invention is directed to a composition comprising PARPi-fl, wherein fl comprises a fluorophore, for use in an intraoperative method of in vivo diagnosis of a tumor margin and/or residual tumor tissue in a subject during tumor removal surgery, wherein the in vivo diagnosis comprises: delivering the composition onto and/or into a viewable tissue surface of the subject; flushing the viewable tissue surface to reduce or remove unbound components of the composition while leaving bound components of the composition; and detecting fluorescence from the fluorophore after administration to the viewable tissue surface, thereby identifying the tumor margin and/or residual tumor tissue.

In certain embodiments, the tissue is cancers of the aerodigestive tract, gastrointestinal tract, urinary tract, ovarian cancer, oral cancer, colorectal cancer, stomach cancer, bladder cancer, cervical cancer, retinal cancer, skin cancer, lung cancer, bronchus cancer, esophageal cancer, or any cancer that can be observed close to the tissue surface with a laparoscopic microscope (pancreatic, liver, kidney, spleen) or any cancer that is surgically resected, and where tissue margins can be observed (e.g., brain).

In certain embodiments, the administering is topically administered or intravenously administered.

In certain embodiments, the viewable tissue surface is viewable by direct exposure or by a camera with access to the tissue surface (e.g., via endoscope).

In certain embodiments, the composition is a liquid composition. In certain embodiments, the liquid composition comprises a rinse.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the flushing comprises waiting a period of time following the administering step such that the tissue clears unbound components of the composition (e.g., via the lymphatic system, endocytosis).

In certain embodiments, the flushing comprises rinsing (e.g., with water or saline) and/or gargling (e.g., with water or saline).

In certain embodiments, potential OSCC is identified using a fluorescence scanning imaging device.

In certain embodiments, potential OSCC is identified following exposure of the oral cavity tissue to excitation light.

In another aspect, the invention is directed to a composition comprising a PARP inhibitor conjugated to a fluorophore for use as an imaging agent.

In another aspect, the invention is directed to a composition comprising a PARP inhibitor conjugated to a fluorophore for use in in vivo diagnosis.

In certain embodiments, the PARP inhibitor is selected from the group consisting of AZD2281, AG014699 (rucaparib), ABT888 (veliparib), BSI201 (iniparib), BSI101, DR2313, FR 247304, GPI15427, GPI16539, M 4827, NU1025, NU1064, NU1085, PD128763, PARP Inhibitor H (INH2BP), PARP Inhibitor m (DPQ), PARP Inhibitor VHI (PJ34), PARP Inhibitor IX (EB-47), and TIQ-A.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the composition is a liquid composition. In certain embodiments, the liquid composition is selected from the group consisting of an oral rinse, a mouthwash, a spray, an intravenous injection, and a local intramuscular injection.

In certain embodiments, the composition is a gel, paste, or other solid or spray.

In certain embodiments, the PARPi-fl binds to PARP1 and preferentially accumulates (or only accumulates) in an area of elevated PARP1 expression, thereby signifying OSCC at the area of accumulation.

In another aspect, the invention is directed to a composition comprising a PARP inhibitor conjugated to a fluorophore for use in a method of assessing a cancer therapy in a subject, wherein the method comprises administering the composition to the subject.

In certain embodiments, the PARP inhibitor is selected from the group consisting of AZD2281, AG014699 (rucaparib), ABT888 (veliparib), BSI201 (iniparib), BSI101, DR2313, FR 247304, GPI15427, GPI16539, M 4827, NU1025, NU1064, NU1085, PD128763, PARP Inhibitor H (INH2BP), PARP Inhibitor m (DPQ), PARP Inhibitor VHI (PJ34), PARP Inhibitor IX (EB-47), and TIQ-A.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In another aspect, the invention is directed to a kit comprising: a PARP inhibitor conjugated to a fluorophore.

In certain embodiments, the PARP inhibitor is selected from the group consisting of AZD2281, AG014699 (rucaparib), ABT888 (veliparib), BSI201 (iniparib), BSI101, DR2313, FR 247304, GPI15427, GPI16539, M 4827, NU1025, NU1064, NU1085, PD128763, PARP Inhibitor H (INH2BP), PARP Inhibitor m (DPQ), PARP Inhibitor VHI (PJ34), PARP Inhibitor IX (EB-47), and TIQ-A.

In certain embodiments, the fluorophore comprises a boron-dipyrromethene (e.g., 4,4-difluoro-4-bora-3a,4a-diaza-s-indacene-3-propionic acid or a salt, moiety, or other equivalent thereof).

In certain embodiments, the fluorophore is conjugated to the PARPi or the fl and/or PARPi moiety(ies) are modified to permit conjugation.

In certain embodiments, the PARPi-fl has a molecular weight no greater than about 1000 Da (e.g., no greater than 900 Da, e.g., no greater than 800 Da).

In certain embodiments, the PARPi-fl has at least a moderate lipophilicity (e.g., to permit penetration into a cell nucleus).

In certain embodiments, the fl moiety having a molecular weight no greater than about 500 Da, e.g., no greater than about 400 Da, e.g., no greater than about 300 Da.

In certain embodiments, the kit further comprises ¹⁸F-PARPi.

In another aspect, the invention is directed to a composition comprising PARPi-fl, wherein fl comprises a fluorophore, for use in (a) a method of in vivo diagnosis of cancer in a subject with oral squamous cell carcinoma (OSCC) or (b) a method of assessing a cancer therapy in a subject, wherein the method comprises: delivering the composition onto and/or into tissue in an oral cavity of the subject; optionally, flushing the tissue in the oral cavity of the subject to reduce or remove unbound components of the composition while leaving bound components of the composition; and optionally, detecting fluorescence from the fluorophore after the administering and the flushing steps, thereby identifying potential OSCC.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Headers are provided for convenience only and are not intended to limit the content or applicability of the material thereunder.

“Administration”: The term “administration” refers to introducing a substance into a subject. In general, any route of administration may be utilized including, for example, parenteral (e.g., intravenous), oral, topical, subcutaneous, peritoneal, intraarterial, intramuscular, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. In some embodiments, the composition described herein is a carrier.

“Non-invasive”: As used herein, the term “non-invasive” refers to methods that are non-surgical, e.g. not penetrating the body, as by incision or injection, or not invading tissue.

In some embodiments, topical administration of a composition to a surface of a tissue is understand as a non-invasive technique.

“Subject”: As used herein, the term “subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conduction with the accompanying drawings, in which:

FIGS. 1A-1C show PARPi-fl tumor accumulation in OSCC and PARP1 expression.

FIG. 1A shows representative images of tumor, muscle and tissues of the oral cavity 2 h after injection of PARPi-fl (100 μL, 2.5 mg/kg) or vehicle only, imaged ex vivo.

FIG. 1B shows fluorescence quantification of tumor, muscle and tongue tissue.

FIG. 1C shows PARP1 Western Blot of imaged organs.

FIGS. 2A-2D show an confocal tumor endoscopy of oral cancer and healthy tongue tissue using PARPi-fl as a fluorescent contrast agent, which is an embodiment that can be used to characterize the invention.

FIG. 2A shows an endomicroscopy imaging system, which is an embodiment that can be used to characterize the invention.

FIG. 2B shows an endoscopic imaging probe with a 1.1 mm diameter, which is an embodiment that can be used to characterize the invention.

FIGS. 2C and 2D shows fluorescence imaging of a (C) healthy tongue tissue and (D) OSCC tumor tissue after intravenous injection of PARPi-fl (2.5 mg/kg, 100 μL). Scale

bar: 50 μm.

FIG. 3A shows detection of PARPi-fl with a prototype dual axis confocal (DAC) microscope, which is an embodiment that can be used to characterize the invention.

FIG. 3B shows a handheld DAC imaging system that can be used for an embodiment of the invention.

FIGS. 3C and 3D show the performance of a desktop DAC microscope, demonstrating high-resolution imaging of fresh tissues injected with PARPi-fl. There is high nuclear staining in tumor tissues, whereas uptake in healthy tissues is low, resulting in high tumor-to-normal contrast ratios.

FIG. 3E shows reconstruction of a Z-Stack of images of normal mouse tongue 90 minutes after PARPi-fl injection. No nuclear accumulation can be seen. The only visible signal is produced by the papillae on the tongue surface of mice.

FIG. 4 shows PARP1 expression in mouse models oral cancer. In both orthotopic and xenograft mouse models of oral cancer, the expression of PARP1 is significantly upregulated in tumor tissue versus the healthy surrounding tissues.

FIGS. 5A-5D show properties and binding of AZD2281-FL.

FIG. 5A shows a binding model depicting olaparib and PARP-1, with the 2H-phthalazin-1-one binding to the catalytically active site of PARP1.

FIGS. 5B-5D show a structure of olaparib, (C) PARPi-fl and (D)¹⁸F-PARPi; 1(2H)-phthalazinone-based PARP-1 binding functionality, (blue); BODIPY FL, the fluorescent reporter (orange); PET active ¹⁸F-label (yellow circle).

FIG. 6 depicts intracellular consequences of PARP1 activation. Intracellular DNA damage leads to PARP1 activation and PARP1-mediated DNA repair.

FIG. 7 shows correlation of cellular PARPi-fl uptake and relative PARP expression. In PANC-1 cells, there was an excellent correlation between intracellular PARPi-fl distribution and PARP-1/2 expression: anti-PARP (red), PARPi-fl (green), composite image of anti-PARP and PARPi-fl, and Pearson correlation coefficient of anti-PARP and PARPi-fl.

FIG. 8 shows real-time in vivo drug distribution of PARPi-fl. Following bolus intravenous administration, PARPi-fl perfused the functional tumor vasculature within seconds and extravasated within minutes. The drug initially distributed nonspecifically within H2B-RFP-expressing cells. Rapid target binding (within minutes) combined with clearance of non-specific membrane labeling increased the specificity of target versus nontarget uptake within an hour. Specific nuclear PARP targeting was observed (bottom right) and this was maintained for several hours.

FIGS. 9A-9B show cell population drug kinetics.

FIG. 9A shows in vivo confocal imaging of PARPi-fl in tumor cells.

FIG. 9B shows the average cellular drug concentration in this example is 1.2 μM. Concentration rapidly increases after a bolus dose followed by a slow decay (top). Analyzing the standard deviation of 250 cells over time showed the highest deviation at early time points decreasing to a much lower level as the diffusive gradients dissipated (bottom).

FIGS. 10A-10E show PARPi-fl imaging of orthotopic U87 tumors with and without prior injection of olaparib.

FIG. 10A shows white light, fluorescence, and overlay images of healthy brain and orthotopic tumor-bearing brain imaged with an IVIS spectrum fluorescence imaging system 1 hour post intravenous injection. U87 tumor tissues were injected with either vehicle alone, PARPi-fl (2.5 mg/kg, 200 μL of 19.5% 1:1 DMAC:Kolliphor, 3.5% DMSO, 77% PBS) or olaparib/PARPi-fl (125 mg/kg olaparib in 100 μL of 7.5% DMSO, 12.5% Cremophor, 80% PBS, followed 30 minutes later by 2.5 mg/kg PARPi-fl in 200 μL of 19.5% 1:1 DMAC:Kolliphor, 3.5% DMSO, 77% PBS);

FIGS. 10B-10E show fluorescence microscope imaging of tumor tissues in FIG. 6A, confirming nuclear uptake of PARPi-fl in non-blocked tumor tissues, but not in the vehicle or olaparib pre-treatment groups.

FIGS. 11A-11B show synthesis of ¹⁸F-PARPi.

FIG. 11A shows the inherently present fluorides in the BF₂ group of PARPi-fl can be replaced in the presence of a Lewis acid catalyst with ¹⁸F. This yields a structurally identical PET active fluorescent PARP1 imaging agent.

FIG. 11B shows HPLC MS traces using a γ-counter (red) and a photodiode array detector (green), confirming identity and purity of ¹⁸F-PARPi.

FIGS. 12A and 12B show a comparison of autoradiography (FIG. 12A) and fluorescence (FIG. 12B) imaging with ¹⁻⁸F-PARPi. ¹⁸F-PARPi and injected the material into tumor-bearing xenograft mice (250 Xi, 29.6 GBq/μmol (0.8 Ci/μmol); For sufficient fluorescence signal, 50 nmol of cold carrier material was co-injected). Both Panels show tissues from the same animal.

FIGS. 13A-13B show pharmacokinetics of ¹⁻⁸F-PARPi.

FIG. 13A shows PET and PET/CT images of a tumor-free mouse with after injection of ¹⁸F-PARPi (250±10 μCi, 50 μmol, 100 μL PEG200/PBS=1/10, 60 min post injection).

FIG. 13B shows pharmacokinetic profile of ¹⁸F-PARPi in a tumor-free mouse. Bone uptake amounts for 6% ID/g. While low, imaging in larger mammals typically reduces bone-uptake further due to slower metabolic degradation.

FIGS. 14A-14C show automated synthesis of ¹⁸F-PARPi from ¹⁸F- and PARPi-fl.

FIG. 14A shows a schematic representation of the fluid path for ¹⁸F-PARPi synthesis. While the drying occurs in a separate reactor, the ¹⁸F-PARPi synthesis is a one pot procedure, and requires, including HPLC purification, drying, and reformulation into an injectable vehicle, only 75 min synthesis time.

FIG. 14B shows a Scansys F2 synthesizer (EnglePhysics, Los Alamos, N. Mex.), which is an embodiment that can be used to characterize the invention.

FIG. 14C shows an embodiment of process optimization.

FIGS. 15A-15C show immunofluorescence staining of PARP1 in human oral cancer squamous cell carcinoma xenografts (OSCC).

FIG. 15A shows H&E staining with corresponding PARP1 immunofluorescence microscopy of OSCC xenografts.

FIG. 15B shows H&E staining with corresponding PARP1 immunofluorescence microscopy of normal mouse tongue.

FIG. 15C shows quantification of the PARP1-positive area in relation to the whole tissue area and average intensity of PARP1 staining in all nuclei, for various xenograft and normal mouse control tissues. The PARP1-positive tissue area was determined by performing a thresholding on red (PARP1) and green (autofluorescence of total tissue) areas and the relative PARP1-positive area was calculated by dividing the PARP1-positive area by the total tissue area. Values display means±SD of n=10 values per tumor/organ. See FIGS. 27A-C for images of trachea and thigh muscle, as well as images obtained with a staining control.

FIG. 16 shows detection of an orthotopic OSCC tumor model in mouse tongue. Left: Epifluorescence imaging of tongue tumor (FaDu) bearing and healthy mice that have been injected with 75 nmol PARPi-fl or vehicle 90 min prior to imaging shows accumulation of PARPi-fl only in tumor bearing tongues. Imaging was also performed using a Lumar fluorescence stereoscope which bridges the gap between whole body and microscopic imaging. Images were taken in bright-field and with 488 nm laser excitation. Here, identification of tumoral regions in mouse tongues with a high contrast to surrounding tissue. The fluorescence images are displayed as grayscale and color-thresholded images to illustrate the accumulation of PARPi-fl (max signal: white, min signal: black).

FIG. 17 shows that after PARPi-fl injection and in vivo imaging, tumor bearing and healthy tongues were cryopreserved, sectioned and imaged with a confocal microscope to detect PARPi-fl following fixation and Hoechst staining of nuclei. H&E staining in adjacent sections shows specific accumulation of PARPi-fl only in areas of tumor tissue but not normal tongue.

FIGS. 18A-18E show PARP1 expression in human tongue tumors.

FIG. 18A shows surgically removed human squamous cell carcinoma specimens of the tongue were stained for PARP1 using an anti-PARP antibody and immunohistochemical detection. Adjacent sections were stained with H&E for pathological evaluation and staging of the malignancy of the tissue. The dotted line marks the tumor margin in the displayed T2 staged tumor. The red squares indicate enlarged regions in adjacent images. The image shown is a representative example selected from n=12 samples.

FIG. 18B shows PARP1 quantification in human tongue tumor tissue in a waterfall plot of the PARP1-positive tissue area. The PARP1-positive tissue is represented by brown (PARP1) and the total tissue areas by blue staining. In each sample, 5-10 fields of view in the tumor area or adjacent healthy tissue were analyzed. Displayed are means±SD. HTT=human tongue tumor.

FIG. 18C shows PARP1-positive tissue area, grouped for normal tissue, premalignant, and malignant (T2-T4 tumor stages) cases. Statistical significance was determined using an analysis of variance where multiple comparisons were controlled using the Holm-Šidák at the familywise error rate of 5%.

FIG. 18D shows individual values for PARP1-positive tissue area, grouped for the pathologically assessed tumor stage (premalignant, T2, T3, T4) and compared to normal adjacent tissue.

FIG. 18E shows density plot of all PARP1-positive tissue area values from each field of view, pooled in two groups (normal and remalignant/malignant). See FIGS. 26A-26C for additional statistical parameters.

FIG. 19 shows PARP1 quantification in human tongue tumor tissue. A PARP1 positive area was determined by performing a thresholding on brown (PARP1) and blue (total tissue) areas and the relative PARP1 positive area was calculated by dividing the brown area by the blue area. In each sample, 10 fields of view in the tumor area and 6 fields of view of adjacent healthy tissue were analyzed. Displayed are means±SD.

FIGS. 20A-20E show PARPi-fl accumulation in OSCC xenografts in mice.

FIG. 20A shows molecular structure and physicochemical properties of PARPi-fl.

FIG. 20B shows representative epifluorescence images of FaDu tumor, tongue, muscle, and trachea. Radiant efficiency displayed in units of [(photons/s/cm²/sr)/(μW/cm²)].

FIG. 20C shows average radiant efficiency of FaDu tumors and tongue.

FIG. 20D shows tumor/organ-to-muscle ratios from images of FaDu and Ca127 tumors, tongue, and trachea calculated as the average radiant efficiency in a region of interest.

FIGS. 20C and 20D display means and SD from n≥5 tissue specimens for PARPi-fl, olaparib/PARPi-fl, and n≥3 for vehicle control. Images and semiquantitative analyses of fluorescence intensities were acquired ex vivo 90 minutes post-injection of PARPi-fl (75 nmol/167 μl PBS with 30% PEG300), Block (olaparib; 3750 nmol/100 μl PBS with 30% PEG300 30 minutes prior to PARPi-fl), or vehicle (167 μl PBS with 30% PEG300).

FIG. 20E shows confocal images of PARPi-fl signal in freshly excised tumor, tongue, and trachea at 90 minutes post-injection of PARPi-fl or Block (as described in FIG. 20A).

FIGS. 21A-21B show localization of PARPi-fl in relation to cell nuclei and PARP1 protein using confocal imaging.

FIG. 21A shows cell nuclei that were stained (ex vivo) with Hoechst 33342 (blue), fluorescence from PARPi-fl injected intravenously in vivo, and PARP1 that was stained with an anti-PARP1 antibody (ex vivo) and detected via immunofluorescence.

FIG. 21B shows a correlation analysis (Pearson R squared) between intravenously injected PARPi-fl and PARP1 immunofluorescence in FaDu tumor tissue, based on the intensity of green or red fluorescence in the nuclear area. The nuclear area was determined via a threshold based on blue fluorescence (Hoechst). A total of 222 correlation pairs were pooled from n=3 tumor sections.

FIGS. 22A-22D show detection of an orthotopic OSCC xenograft model in mouse tongue.

FIG. 22A shows epifluorescence imaging of tumor-bearing tongues (FaDu) and healthy mouse tongues from animals injected with PARPi-fl (75 nmol/167 μl PBS with 30% PEG300) or vehicle (PBS with 30% PEG300) 90 minutes prior to imaging.

FIG. 22B shows imaging with a Lumar fluorescence stereoscope. Images were taken in brightfield and with 488 nm laser excitation. Fluorescent images are displayed in grayscale and intensity-scaled (“+”=maximum signal, “−”=minimum signal).

FIG. 22C shows cryopreserved tongue sections were imaged with a confocal microscope to detect PARPi-fl following fixation. H&E staining in adjacent sections for anatomical and pathological evaluation of PARPi-fl localization.

FIG. 22D shows average radiant efficiency [p/s/cm²/sr]/[μW/cm²] of PARPi-fl in excised tumor bearing and healthy tongues as well as trachea and thigh muscle. Means and SD from n=3 animals/group are displayed.

FIGS. 23A-23E show microscopic imaging of whole excised FaDu tumors.

FIG. 23A shows whole excised FaDu tumor and mouse tongue were imaged 90 minutes post-injection of PARPi-fl (75 nmol/167 μl PBS with 30% PEG300) using a custom-built dual-axis confocal microscope with 488-nm laser excitation.

FIG. 23B shows reconstruction of a Z-Stack spanning a depth of 250 μm to show PARPi-fl nuclear localization.

FIG. 23C shows whole excised FaDu tumors were also imaged using a commercial confocal laser endomicroscope featuring a flexible microprobe with a resolution of 1.4 μm.

FIG. 23D shows FaDu tumor, mouse tongue, and muscle were imaged 90 minutes post-injection of PARPi-fl (75 nmol/167 μl PBS with 30% PEG300) following sacrifice of the animals. Images are representative frames within real-time video recordings of the organs at 488-nm laser excitation. Identical window/leveling has been applied in all images.

FIG. 23E shows analysis of signal intensity after PARPi-fl or vehicle injection. Ten frames per specimen were analyzed for their average pixel intensity using ImageJ (n=3). Statistical significance was determined using an unpaired t-test, corrected for multiple comparisons by the Holm-idak method with an alpha of 0.05.

FIGS. 24A-24D show oral cancer delineation after topical application of PARPi-fl.

FIG. 24A shows an exemplary imaging procedure for OSCC detection using a PARPi-fl based, orally applied solution. Imaging of the oral cavity with a fluorescence camera can be conducted after a one minute topical PARPi-fl application, followed by removal of unbound or nonspecifically bound compound with the clearing solution 1% acetic acid. The illustration is courtesy of MSKCC (2015).

FIG. 24B shows spectrally resolved epifluorescence imaging of healthy mice and tongue tumor bearing mice before and after topical application of PARPi-fl. The tdTomato signal indicates the position of the tumor, while the PARPi-fl signal shows the ability of PARPi-fl to specifically detect sites of OSCC after topical application. All images are scaled to the same maximum radiant efficiency.

FIG. 24C shows epifluorescence imaging of healthy mice and mice bearing orthotopic tongue tumors. The tumors express tdTomato fluorescent protein, as control for tumor localization. Imaging of PARPi-fl signal was conducted before and after PARPi-fl topical application.

FIG. 24D shows confocal microscopy of an OSCC bearing tongue after topical application of PARPi-fl in vivo. H&E confirms presence of tumor. Arrows point to enlarged images of the squared area.

FIG. 25 shows PARP1 staining of human oral cancer tissues. Representative PARP1 staining of different degrees of malignancy of human oral cancer tissue (upper row) and adjacent normal tongue tissue (lower row). PARP1 immunohistochemical staining was conducted on formalin-fixed, paraffin-embedded surgically removed tissues of human squamous cell carcinoma of the tongue using an anti-PARP antibody and IHC detection.

FIGS. 26A-26C show PARP1 expression and statistics of human oral cancer tissues.

FIG. 26A shows PARP1 quantification in human tongue tumor tissue displaying the PARP1-positive tissue area in %. For each specimen, malignant tissue and corresponding healthy adjacent tissue is shown. The PARP1-positive tissue area was determined as described in the methods section. For each sample, 5-10 data points in the tumor area or adjacent healthy tissue were analyzed. Displayed are means±SD of all samples. HTT=human tongue tumor; color code: dashed stripes=malignant tissue (squamous cell carcinoma T2-T4), solid stripes=premalignant cases (moderate to severe dysplasia, carcinoma in situ), white=corresponding normal adjacent tongue tissue.

FIG. 26B shows that the performance of PARP1 as a classifier for tumor and normal tissue was evaluated using a receiver operating characteristic (ROC) curve.

FIG. 26C shows that the probability of a given tissue being malignant as a function of the PARP1-positive tissue area (in percent) was estimated by nonparametric binary regression using the method of local likelihood.

FIGS. 27A-27B show PARP1 expression in subcutaneous mouse models of human oral cancer.

FIG. 27A show H&E staining with corresponding PARP1 IF staining of OSCC xenografts and mouse control tissues tongue, trachea, and muscle. Scale bar: 50 μM.

FIG. 27B shows control of staining specificity. The red PARP1 signal disappears when a nonspecific rabbit IgG is used instead of the anti-PARP1 primary antibody, showing the specificity of the secondary antibody for binding to the primary antibody. Scale bar: 50 μM.

FIGS. 28A-28D show co-localization of PARP1 and PARPi-fl in vivo. Co-localization of PARP1 antibody staining with in vivo injected PARPi-fl in cryosections of FaDu xenografts.

FIG. 28A show anti-PARP1 staining co-localized with PARPi-fl and both show a nuclear localization.

FIG. 28B show no non-specific binding of the secondary antibody to the tissue occurred since the PARP1 signal disappeared when PBS was used instead of the anti-PARP1 primary antibody.

FIG. 28C show that when a rabbit IgG was used as isotype control instead of the anti-PARP1 antibody, only a very weak red staining was observed, showing minimal non-specific binding of the secondary antibody to the IgG.

FIG. 28D show that when sections were only stained for PARP1, but no PARPi-fl was injected in vivo, there was no nuclear signal, showing that no bleedthrough of signals through the fluorescence channels occurred. The images were taken on a Leica SP8 inverted confocal microscope. Scale bar: 50 μM.

FIG. 29 shows PARP1 expression in an orthotopic mouse model of oral cancer. PARP1 staining in an orthotopic mouse model of oral cancer (derived from FaDu cells) with H&E staining of the corresponding areas. The PARP1 expression in the tumor area is much higher than in the surrounding normal tongue muscle and mucosal tissue. PARP1 immunohistochemical staining was conducted on formalin-fixed, paraffin-embedded tumor-bearing tongues of nude mice using an anti-PARP antibody and IHC detection.

FIG. 30 shows imaging of PARPi-fl accumulation using a fluorescence endoscope. Whole excised FaDu tumors were imaged using a confocal laser endomicroscope featuring a flexible confocal microprobe with a resolution up to 1.4 μm. FaDu tumor, mouse tongue, and muscle have been imaged 90 minutes post-injection of 150 nmol PARPi-fl (30% PEG300 in PBS) following sacrifice of the animals. Images are representative frames within real-time video recording of the organs at 488 nm laser excitation. Same window/leveling has been applied in all images. The fluorescence signal has been converted to an intensity scale.

FIGS. 31A-31C show PARP1 expression in oral squamous cell carcinoma.

FIG. 31A shows PARP1 Immunohistochemical staining of FaDu and Ca127 xenografts as well as normal mouse tongue and H&E staining of adjacent sections.

FIG. 31B shows quantification of the PARP1 positive area in FaDu, Ca127 and tongue tissue.

FIG. 31C shows high magnification images of specimens displayed in FIG. 31A.

FIGS. 32A-32D show effects of irradiation on cell survival and PARPi-fl uptake.

FIG. 32A shows examples of clonogenic growth of FaDu and Ca127 cells without (0 Gy) and after 10 Gy irradiation.

FIG. 32B shows clonogenic survival curve of FaDu and Ca127 cells after 0, 2, 4, 6, 8 and 10 Gy irradiation. Means±SEM of three independent experiments with three parallels each. The asterisk indicates a p-value<0.05 between FaDu and Ca127 cells using Student's t-test.

FIG. 32C shows nuclear PARPi-fl uptake without and after 10 Gy irradiation, as observed microscopically in FaDu cells.

FIG. 32D shows Ca127 cells. Nuclear localization was confirmed by Hoechst DNA stain. Representative images from n=3 experiments.

FIGS. 33A-33D show quantification of PARPi-fl uptake after irradiation. Specific uptake of PARPi-fl into FaDu (FIG. 33A) and Ca127 (FIG. 33B) cells was determined by flow cytometry after incubation of cells with PARPi-fl or olaparib/PARPi-fl. Olaparib competes with PARPi-fl for specific binding sites on PARP1. PARPi-fl uptake of FaDu (FIG. 33C) and Ca127 cells (FIG. 33D) after irradiation with 2, 4 and 10 Gy was determined and compared to 0 Gy. Mean PARPi-fl fluorescence signal was measured via flow cytometry in the FITC channel. Bars represent means±SEM from three independent experiments with three parallels each.

FIG. 34 shows experimental design for in vivo irradiation. Bilateral subcutaneous FaDu xenografts were grown for 15 days before 10 Gy irradiation of the tumor on the right flank. Subsequently, the effect of the irradiation on PARP1 expression and PARPi-fl uptake was observed 24 hours and 48 hours post irradiation and compared between the non-irradiated and the irradiated tumor. The effect of irradiation on tumor growth was monitored until day 26 post tumor inoculation or until tumors exceeded a volume of 1000 mm³.

FIG. 35 shows growth curves of subcutaneous FaDu tumors over 26 days. Tumors were irradiated with 10 Gy using an image-guided microirradiator (day 15). Controls (0 Gy) were not irradiated (n≥4/group). The asterisk indicates statistical significance with p<0.05 using the Student's t-test.

FIGS. 36A and 36B show PARP1 Immunofluorescence staining of irradiated and non-irradiated tumors.

FIG. 36A shows representative PARP1 Immunofluorescence staining (n=4/group). Red: PARP1; blue: Hoechst DNA stain.

FIG. 36B shows quantification of the PARP1 intensity per nucleus and the PARP1 positive area of irradiated (10 Gy) tumors in relation to non-irradiated (0 Gy) tumors. Values are based on quantification in 10 fields-of-view per tumor and four tumors per time point. Displayed are means with SEM (normalization was done for each individual before calculating the means).

FIGS. 37A-37C show epifluorescence imaging of PARPi-fl uptake post irradiation.

FIG. 37A shows representative epifluorescence images of excised FaDu xenografts and mouse tongues. Displayed are fluorescence only, photograph only and composite images.

FIG. 37B show quantification of PARPi-fl uptake into non-irradiated (0 Gy) and irradiated (10 Gy) xenografts and mouse tongue (n=5/time point). Shown are means±SD. Asterisk indicates a p-value of <0.05 using Student's t-test.

FIG. 37C shows representative confocal images of the tumors displayed in FIG. 37A.

FIGS. 38A-38B shows specificity control of PARP1 Immunofluorescence staining of irradiated and non-irradiated tumors. Subsequent cryosections of the same tumors were either stained for PARP1 (FIG. 38A) or the primary anti-PARP1 antibody was replaced with a nonspecific rabbit IgG (FIG. 38B) to assess the extent of nonspecific binding. The secondary goat anti-rabbit antibody was labeled with an AF594 red fluorescent dye. In combination with the primary rabbit anti-PARP1 antibody, nuclear staining can be observed which is absent in the rabbit IgG control. Furthermore, no non-nuclear red fluorescent signals were observed, indicating that no measurable non-specific binding was induced by the irradiation.

FIGS. 39A-39B show PARPi-fl ex vivo imaging specificity control.

FIG. 39A shows a comparison of the green fluorescence signal (intensity and histogram distribution) in FaDu tumor tissue with and without PARPi-fl injection to assess the potential impact of autofluorescence.

FIG. 39B shows PARP1 staining of cryosections of a FaDu tumor 48 hours after 10 Gy irradiation to show colocalization between PARPi-fl and PARP1 including specificity controls for the PARP1 staining (replacement of the specific primary anti-PARP1 antibody with rabbit IgG or no primary).

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Described herein are methods of using radiolabeled or fluorescent poly(ADP-ribose)polymerase 1 (PARP1) imaging probes with high selectivity and specificity to detect the cancer biomarker PARP1 in the oral cavity via topical application, e.g. in a dentist office setup using a macroscopic fluorescence scanning imaging device. After topical application of PARPi-fl to the oral cavity, the imaging probe accumulates only in areas of elevated PARP1 expression. Employment of a microscopic device, such as a fluorescence endoscope or a handheld confocal microscope can improve identification of tumor margins and residual tumor tissue during tumor removal surgery.

Earlier and more accurate detection of oral squamous cell carcinoma (OSCC) is essential to improve the prognosis of patients and to reduce the morbidity of surgical therapy. It was demonstrated herein that PARP1 is a target for optical imaging of OSCC with the fluorescent dye PARPi-fl. In patient-derived OSCC specimens, PARP1 expression was increased 7.8±2.6-fold when compared to normal tissue. Intravenous injection of PARPi-fl allowed for high contrast in vivo imaging of human OSCC models in mice with a surgical fluorescence stereoscope and high-resolution imaging systems. The emitted signal was specific for PARP1 expression, demonstrating that PARPi-fl can be used as a topical imaging agent, spatially resolving the orthotopic tongue tumors in vivo. The results suggest that PARP1 imaging with PARPi-fl can enhance the detection of oral cancer, serve as a screening tool and help to guide surgical resections.

Vital signs can be monitored before PARPi-fl administration and after completion of imaging. The oral mucosa can be checked for local irritation, after completion of imaging and up to about 3 days later. Two blood samples can be obtained prior to PARPi-fl imaging and up to about 3 days after PARPi-fl administration.

Radiolabeled and optically active ¹⁸F-PARPi and PARPi-fl were described in “Compositions and methods for in vivo imaging” by Keliher et al., in International Publication Number WO/2012/074840 A2, which is hereby incorporated by reference (See Appendix A). These imaging probes possess favorable pharmacokinetic properties to detect and interrogate tumor growth and treatment success. Moreover, they are compatible with current widely used imaging technologies in laboratory and diagnostic medicine— PET and fluorescence imaging.

As described herein, PARPi-fl and ¹⁸F-PARPi are pharmacologically identical agents that accumulate quickly and selectively in cancer cells that overexpress PARP1, and can therefore serve as a screening tool to noninvasively delineate the presence and extent of neoplastic growth in the oral cavity. The functions of PARPi-fl and ¹⁸F-PARPi are complimentary. Although ¹⁸F-PARPi lacks the high resolution and sensitivity of optical imaging, it enables detection of deep seated lesions such as lymph node and distant metastases. Complimentary to ¹⁸F-PARPi, PARPi-fl highly permeates the cells, providing more accurate (sensitive and specific) detection of oral cancer based on the high PARP1 expression of oral squamous cell carcinoma cells. Furthermore, as disclosed herein, the use of PARPi-fl enables cellular-resolution point-of-care imaging and does not require expensive infrastructure and specialized personnel to accurately and non-invasively screen for lesions with high spatial accuracy. Used together, PARPi-fl and ¹⁸F-PARPi are highly sensitive and specific diagnostic tools that can be used to detect oral squamous cell cancers. Thus, the two orthogonal imaging modalities (e.g. PET and optical imaging) enable screening (e.g., via optical imaging) and staging (e.g., via PET imaging) of this disease.

The PARP1 imaging agents disclosed herein can be used as diagnostic markers for the early detection of oral cancer in the oral cavity (e.g., of a human). The fluorescent PARPi-fl can be used as an optical imaging agent for the screening and diagnosis of squamous cell carcinoma of the oral mucosa (e.g., of a human). Moreover, the ¹⁸F-PARPi can be used as a quantitative PET imaging agent to assist non-invasive diagnoses.

Various mouse models, described herein, have been used to quantify PARP1 expression in OSCC. For example, xenografts and orthotopic mouse models of oral cancer, induced by the injection of human OSCC cancer cells into the tongue bed, have been studied as described herein. Moreover, a model can rely on chemically induced oral cancer (e.g. addition of 4-nitroquinoline 1-oxide to drinking water) or a blinded study can be performed to determine the sensitivity and specificity of PARPi-fl for oral cancer tissue.

PARPi-fl and ¹⁸F-PARPi can be used in human cell line models of OSCC including subcutaneous xenografts, orthotopic xenografts, and chemically induced oral cancer. The pharmacokinetics of PARPi-fl and ¹⁸F-PARPi are also disclosed herein. In some embodiments, agarose phantoms, xenografts and orthotopic models of OSCC for epifluorescence imaging, autoradiography, and PET imaging can be used to determine the correlation curves and quantitative analysis for these imaging techniques.

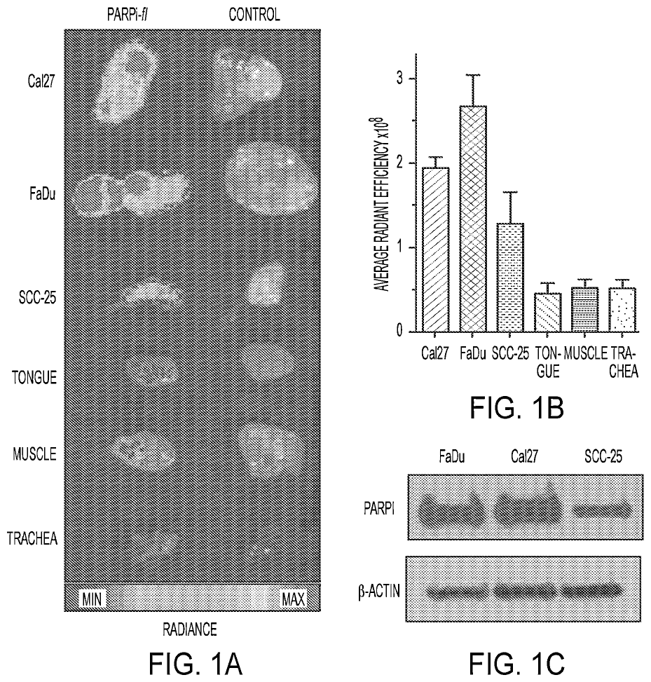

As shown in in FIGS. 1-3, oral squamous cell carcinoma (OSCC) cell lines highly overexpress PARP1, allowing the detection of cancerous lesions. OSCCs develop in the oral mucosa, at the tissue surface.

An in vivo administered dose of ¹⁸F-PARPi and/or PARPi-fl can confirm malignant lesions based on the high expression of PARP1 in highly proliferative tissue. As described herein, PARPi-fl is a valuable tool for the detection of OSCCs in cancer. Moreover, this agent can be used to screen for developing premalignant lesions. Furthermore, its sister imaging agent, ¹⁸F-PARPi, can be a powerful tool to quantify the extent of malignant growth below the tissue surface, at local or even distant sites. A therapeutic PARP1 inhibitor can also be administered with a PARP1 imaging agent. The therapeutic PARP1 inhibitor can be AG014699 (rucaparib), ABT888 (veliparib), BSI201 (iniparib), BSI101, DR2313, FR 247304, GPI15427, GPI16539, M 4827 NU1025 NU1064, NU1085, P1)128763, PARP Inhibitor H (INH2BP), PARP Inhibitor m (DPQ), PARP Inhibitor VIII (PJ34), PARP Inhibitor IX (EB-47), and TIQ-A, as described by Keliher et al.

Therefore, use of these bimodal imaging probes facilitate diagnosis of this cancer which enables adoption of the technology by healthcare professionals.

### Evaluation of the Translational Potential of PARP1 as Part of a Clinical Biopsy Study.

Although the genetic and molecular basis of cancer and its diagnosis via non-invasive imaging has advanced significantly over the last several decades, early diagnosis and noninvasive detection of OSCC remain limited. It appears that no models of human cancer in the oral cavity have been heretofore validated.

It is described herein that PARP1 is a specific and selective early biomarker for the detection of OSCC. Primary human biospecimen of oral cancer were obtained and analyzed using standard clinical pathology and grouped into healthy, premalignant and malignant tissues. Part of the biopsied tissue were used to determine the PARP1 expression. This provided corroboration of to what degree PARP1 expression is elevated in human oral cancer.

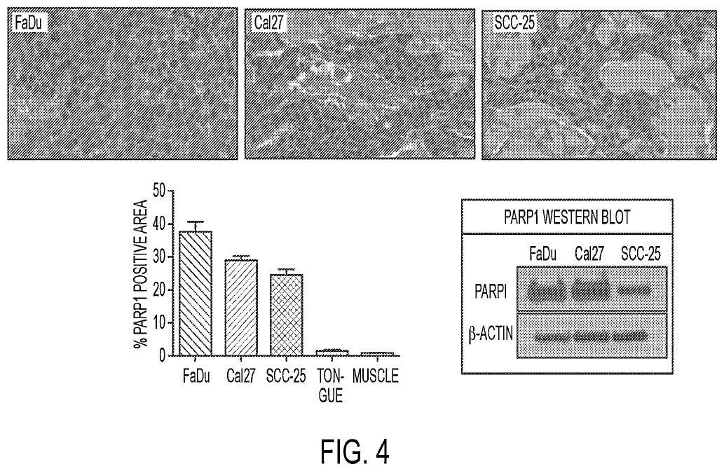

PARP1 expression is highly upregulated in mouse models of oral cancer as shown in FIG. 4. Because more than ninety percent of all oral cancers are squamous cell carcinoma, which arise at the tissue surface of the oral cavity, no deep tissue penetration is therefore necessary to confirm the presence of this type of cancer, which can be identified with targeted fluorescent probes. A fluorescence-based screening technology has the advantage of requiring only little infrastructure. Screening for oral cancer can be achieved using the fluorescent PARPi-fl. For suspected or confirmed cases of oral cancer, however, there is a need to determine the total tumor burden, metastatic and lymphatic involvement. Here, the radiolabeled version of an imaging agent, ¹⁸F-PARPi, can be used for quantification.

Similar to most therapeutic small molecules, ¹⁸F-PARPi and PARPi-fl bind to the NAD⁺ binding site of PARP1. The imaging agents can therefore be used as companion imaging agents for PARP1 inhibitors that are binding to the same location (e.g. ABT-888, Abbott; AG014699, Pfizer; AZD2281, Astra-Zeneca; BMN-673, Biomarin; MK-4827, Merck). PARP1 imaging allows physicians to stratify patients in their appropriate treatment groups, enabling clinical decision making processes based on PARP1 levels.

In combination, these bimodal imaging agents can be used to leverage the unique properties and selective accumulation of these small molecules in proliferative growths. Therefore, when in clinical use, the optical component of PARPi-fl can be used to screen for the presence of oral cancer (which in more than 90% of all cases occurs direct at the tissue surface. Once suspected or confirmed, the PET component of ¹⁸F-PARPi can be used to quantify the exact tumor burden and determine whether the cancer is local or has metastasized.

The fluorescence signal for PARPi-fl stems from the fluorophore. For example, in the case where the fluorophore is BODIPY®-FL, the dye BODIPY-FL emits fluorescent light of wavelength of 525 nm when excited at 488 nm. The PARPi-fl composition has a similar binding affinity to PARP1 as the olaparib ((IC50 for inhibition of PARP1 enzymatic activity 12.5 nM vs. 6.0 nM for olaparib).

### Point-of-Care In Vivo Microscopy

In addition to PET imaging, confocal microscopy of the oral cavity can be used to image the bimodal agent PARPi-fl for fluorescence in vivo. Although PET is a highly sensitive imaging modality for whole-body screening, it requires large infrastructure and lacks the ability to image suspicious lesions noninvasively at the sub-cellular level. Moreover, while wide-field imaging is beneficial for rapid surveillance of an entire oral cavity, it generates a large number of “false-positive” results obtained by such wide-field approaches.

Thus, a miniature, portable confocal microscope that rapidly obtains images of glandular, cellular, and nuclear detail for diagnosing suspicious tissues in vivo, and guides the acquisition of excisional biopsies can be used for screening and detecting tumors in the oral cavity. This optical sectioning technology can both improve the early detection of oral cancers, as well as significantly reduce the time, cost, and patient discomfort associated with the acquisition of large numbers of unnecessary biopsies.

### EXPERIMENTAL EXAMPLES

### Example 1: In Vitro and In Vivo Imaging of PARPi-fl

### PARP1 Imaging Agents

Expression levels of PARP1 are in the micromolar range and thus higher than of many other proteins upregulated in cancer.

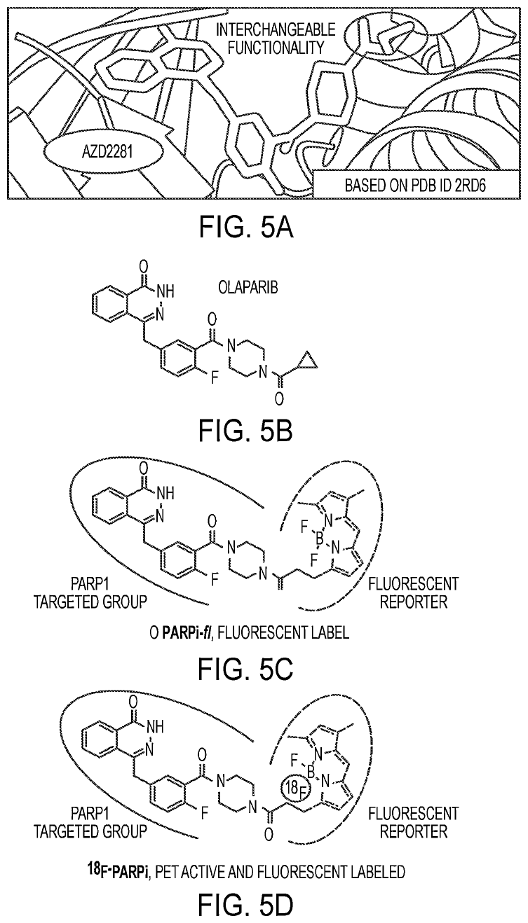

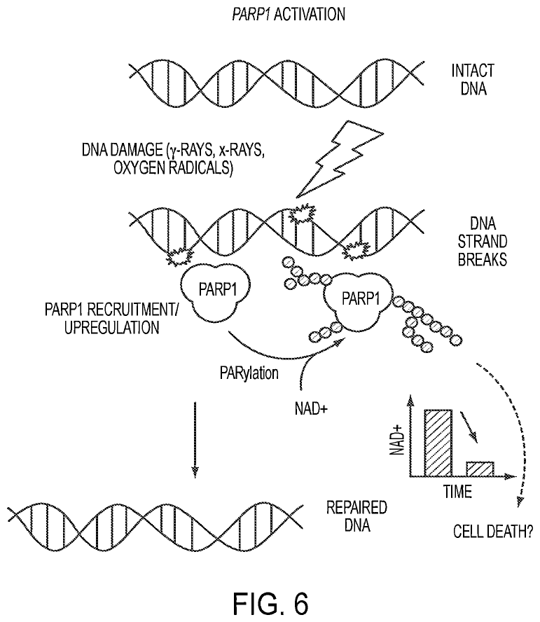

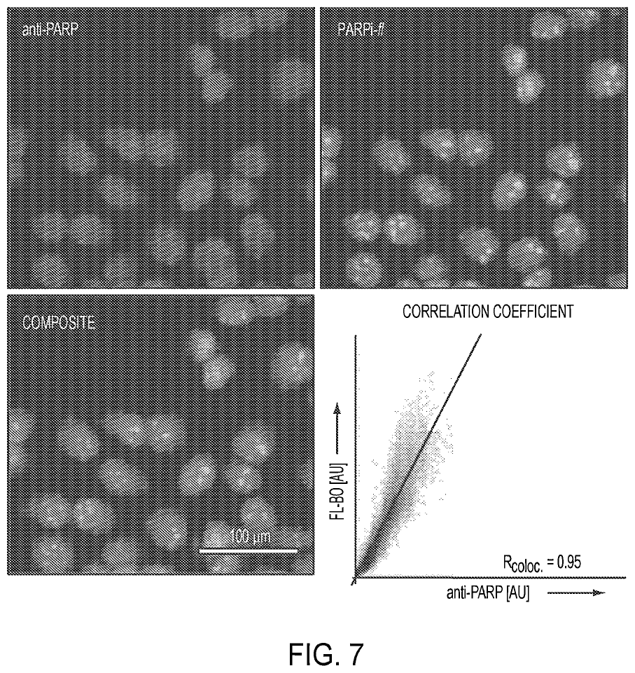

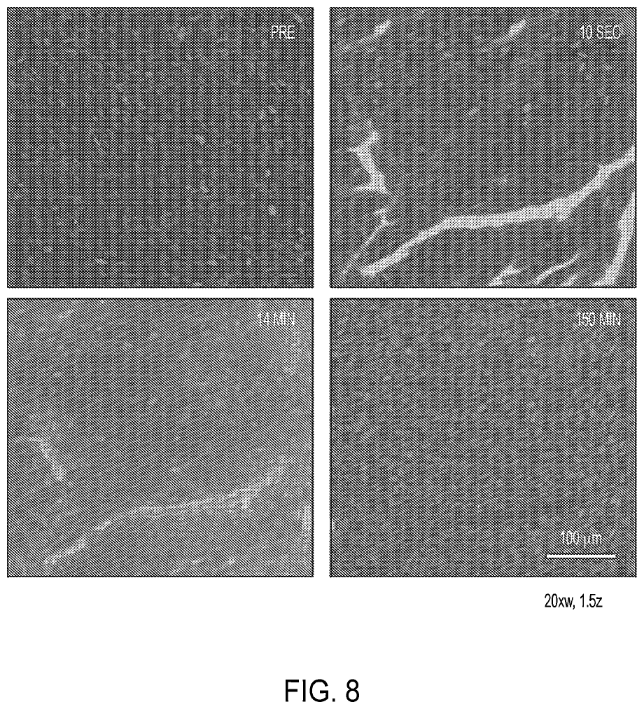

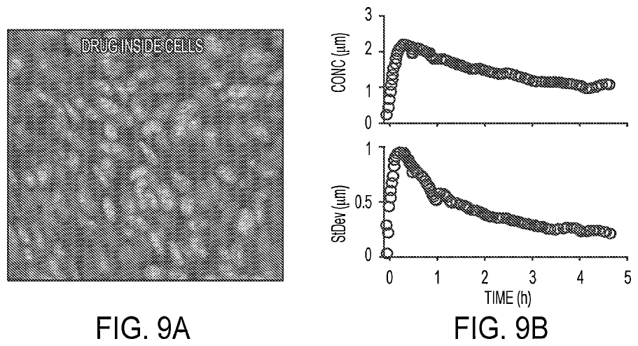

FIGS. 5, 7, and 8 show that, when injected intravenously, PARPi-fl quantifies PARP1 in vivo at sub-cellular resolution as soon as 90 minutes after injection of 75 nmol of PARPi-fl. As shown in FIGS. 9A-9B, PARPi-fl demonstrates a remarkable homogeneous nuclear distribution in the tumor tissue. For example, the uptake of PARPi-fl within tumor tissue illustrates that an overwhelming amount of cells (e.g., greater than 99.8%) are targeted at high concentrations (e.g. 1.9±0.5 μM). Expression of PARP1 in malignant growth is upregulated compared to healthy tissue, and squamous cell carcinoma of the mouth can be detected with PARPi-fl.

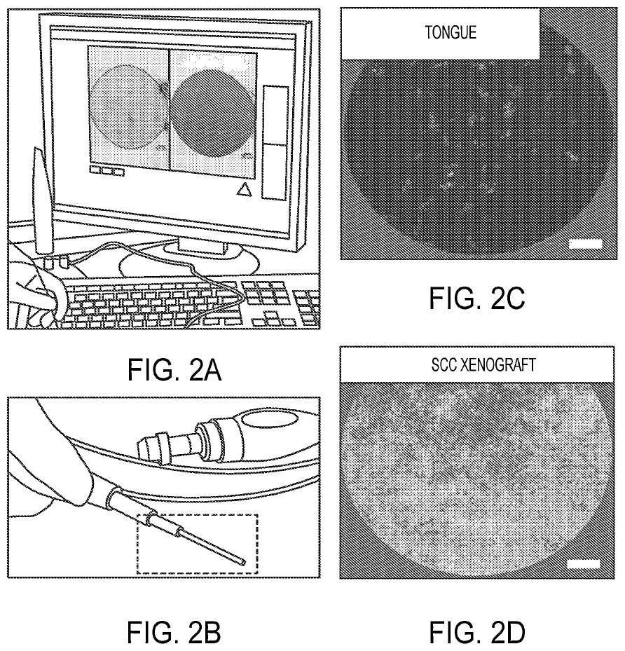

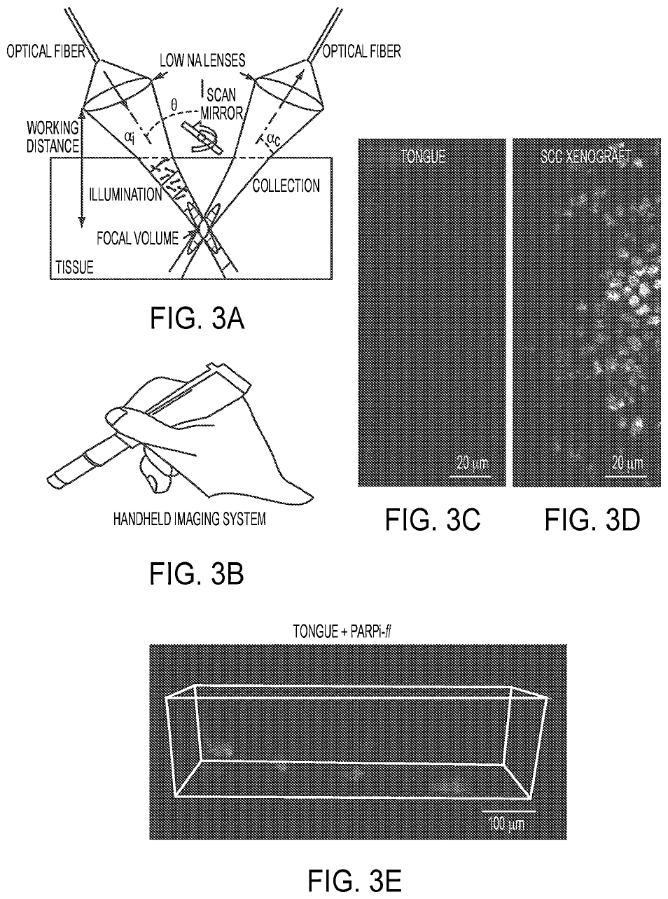

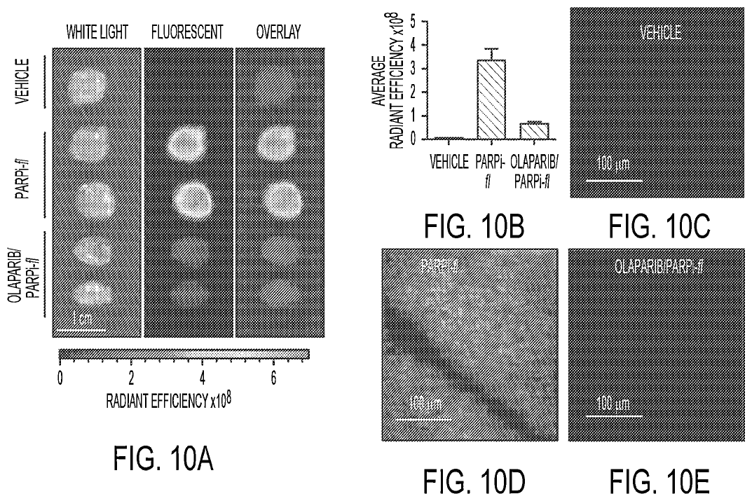

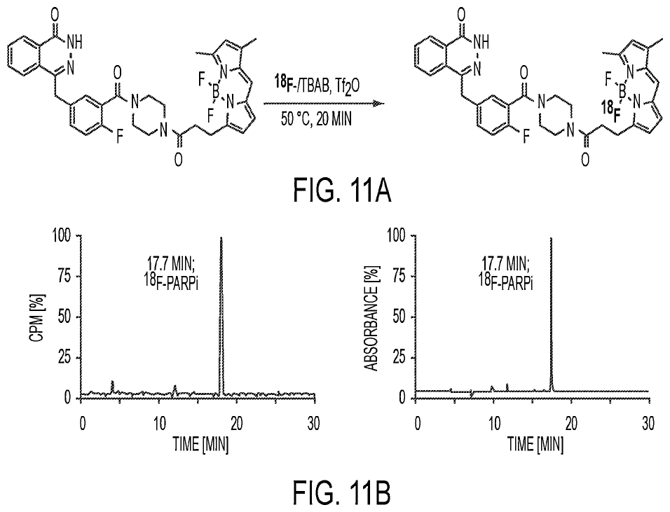

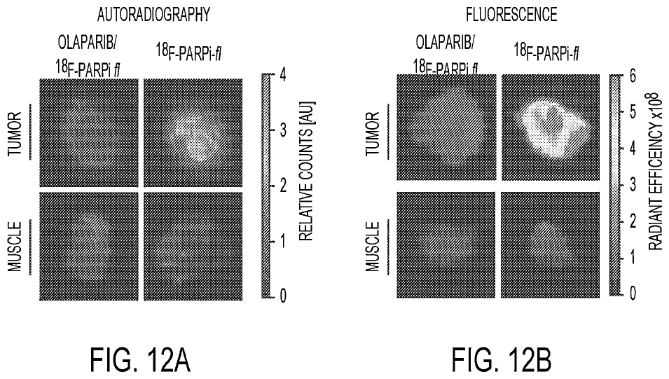

Referring back to FIGS. 1A-1C, PARP1 is highly overexpressed in OSCC of the mouth compared to surrounding oral tissues. As shown in FIGS. 2A-2D, a confocal endomicroscope enables detection of PARPi-fl uptake in OSCC. Moreover, as shown in FIGS. 3A-3E, a prototype dual-axis confocal (DAC) microscope can also detect PARPi-fl uptake in OSCC. Compared to traditional single-axis confocal microscopes, DAC microscopes are less affected by scattered light, therefore allowing deeper tissue penetration and higher contrast for high-resolution optical sectioning of tissues. This expression pattern aligns with other types of cancers, where expression in healthy tissue is similarly sporadic, as shown in FIGS. 10A-10E.

### Synthesis of PARPi Imaging Agents

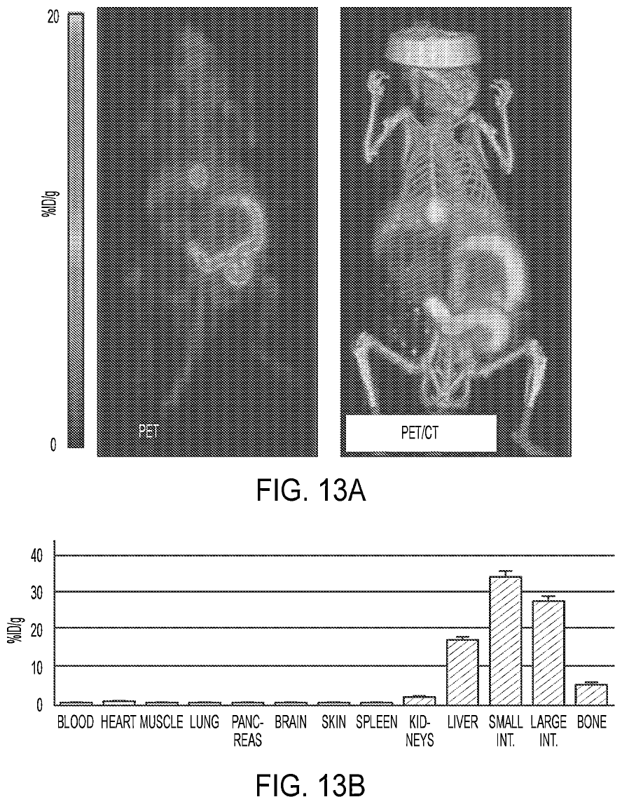

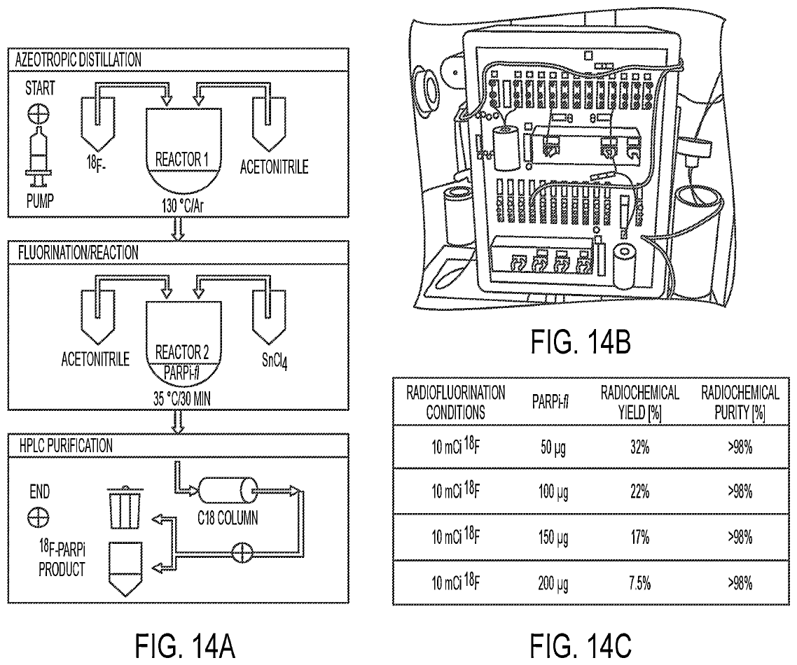

Acid catalyzed ¹⁸F/¹⁹F exchange allows formation of ¹⁸F-PARPi. In the presence of a strong Lewis Acid, a cold ¹⁹F atom attached to BF₂ group of PARPi-fl can be replaced with a PET active ¹⁸F. ¹⁸F-PARPi was synthesized using an automated synthesis module as shown in FIGS. 14A-14C. The pharmacokinetics of ¹⁸F-PARPi in mice were investigated, and it was found that the agent has a favorable pharmacokinetic profile for in vivo imaging as shown in FIGS. 13A-13B. More detail regarding the synthesis of these imaging agents can be found in International publication No. WO/2012/074840 A2 which is hereby incorporated by reference in its entirety.

### In Vitro and In Vivo Imaging of PARPi-fl in Mouse Models of Cancer

Similar to the mouse models of glioblastoma shown in FIGS. 10A-10E, human xenografts of OSCC (e.g. OSCC-4, OSCC-35, HN-OSCC-68, FaDu, Ca127, OSCC-25), were used to determine the uptake of PARPi-fl in comparison to the surrounding healthy tissues (e.g. cheek, tongue, pharynx, larynx, tonsils, esophagus). PARP1 imaging agent were injected intravenously, either with or without prior injection of the non-fluorescent known PARP1 inhibitor olaparib (125 mg/kg of olaparib in 100 μL of 7.5% DMSO, 12.5% Cremophor, 80% PBS, followed 30 minutes later by 2.5 mg/kg PARPi-fl in 200 μL of 19.5% 1:1 DMAC:Kolliphor, 3.5% DMSO, 77% PBS). PARP1 expression was then determined via Western Blot and immunohistochemistry. PARPi-fl uptake was determined using an epifluorescence imaging system.

### Correlation of PARPi-fl Uptake and OSCC in an Orthotopic Oral Cancer Model

Mice were inoculated orthotopically with a fluorescent oral cancer cell line (tdTomato-FaDu and tdTomato-Ca127). PARPi-fl imaging agent uptake in the tumor was observed with an IVIS preclinical imaging system (Perkin Elmer, Waltham, Mass.) and compared to the expression of the fluorescent cell line. A correlation of PARPi-fl and tumor growth can be assumed if the Pearson's correlation coefficient between both fluorescent channels is greater than 0.95.

Blind Detection in Chemically Induced Models of OSCC with PARPi-fl

A chemically induced model of OSCC was used to determine the sensitivity of PARPi-fl for detecting the presence of malignant lesions. For this model, the presence of disease was determined histologically in three categories (cancer, pre-cancer, normal). One hundred mice were used for this purpose. Three separate readers who were blinded to visible light images. Histology results then read the set of fluorescence imaging data in randomized order and determined whether or not, based on a 5-point scale, cancerous tissue was present.

### In Vivo Pharmacokinetics of ¹⁸F-PARPi

The blood half-life, serum stability, and metabolic stability of ¹⁸F-PARPi was determined. Blood half-lives were determined after the injection of ˜15 μCi of the ¹⁸F-labeled species into C57BL/6J (B6) mice and blood (5-10 μL) drawn at different time points via saphenous vein bleeds. FIGS. 13A-13B show an image acquired at 60 minutes. The drawn blood was both counted as well as analyzed using a HPLC-MS equipped with both a fluorescence detector as well as a parallel radiodetector. Organs were homogenized. Next, the bimodal imaging agent was extracted and radioactive/fluorescent metabolites were analyzed using HPLC. All major organs were weighed, and their metabolic activity was measured using a scintillation counter. The injected dose per gram tissue (% ID/g) was determined.

### Determination of Uptake Ratios of ¹⁸F-PARPi in Xenograft and Orthotopic Models of Cancer

The uptake and clearance rates of ¹⁸F-PARPi in xenograft, orthotopic and chemically induced oral cancer was measured. For xenografts, cancer cells (1-5×10⁶ cells in 1:1 PBS:BD Matrigel for mouse xenografts and 5×10⁴ cells for orthotopic models) were injected and the tumors grew for 5-10 days for xenograft models and 12-20 days for orthotopic cancers. For chemically induced OSCC, tumors were induced through the addition of 4-nitroquinoline 1-oxide to drinking water. Comparison of the % ID/g in tumors and healthy oral tissues as well as excretory organs, bone, urine and feces at different injected amounts of the tracer (300 Xi-500 Xi, 0.5 μg-100 μg injected material) were measured at different time points.

In order to assess specificity of the tracer uptake, blocking experiments with both cold PARPi-fl as well as the inhibitor olaparib were designed. Comparison and quantification of PARPi-fl and ¹⁸F-PARPi biodistribution were performed. Agarose phantoms were produced and ¹⁸F-PARPi (at 5 μCi-5000 Xi) and PARPi-fl (0.5 nmol-500 nmol) were imaged to create calibration curves for the bimodal imaging probe system. Imaging of the signal strength using an epifluorescence imaging system, autoradiography, and PET was performed. The resulting calibration curves allowed quantification of the uptake and emission of the imaging agents, and to determine the amount of inhibitor in a given volume. A range of ¹⁸F-PARPi activities (300 mCi-500 mCi) and PARPi-fl concentrations (25 nmol-75 nmol) were injected in mice. The animals were imaged at various time points using all imaging modalities.

### In Vitro and In Vivo Imaging of PARPi-fl in Human Models of Cancer

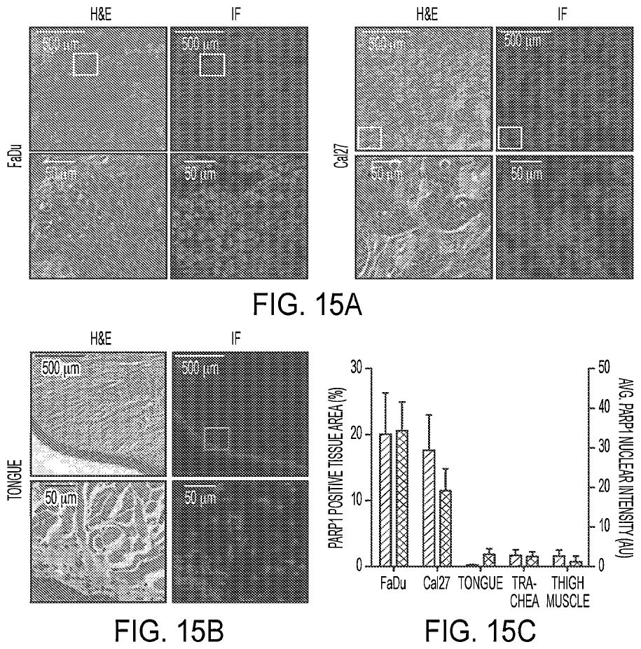

PARP1 expression in human OSCC cell lines in a subcutaneous xenograft mouse model using Immunofluorescence (IF) PARP1 staining is depicted in FIGS. 15A-15C. High expression (PARP1 positive area and intensity of nuclei) in FaDu and Ca127 tumors and low expression in the mouse control tissues tongue, trachea and muscle were found.

The specific uptake of PARPi-fl into these cell lines can be shown in vitro, and can also be confirmed in subcutaneous xenograft models using epifluorescence imaging. Disclosed herein, the accumulation of PARPi-fl in tumor cells but not normal tissue led to high tumor to background ratios (tumor to muscle ratios; FaDu: 4.6±1.4, Ca127: 2.9±1.0). In concordance with a higher PARP1 expression, PARPi-fl accumulation was higher in FaDu than in Ca127. In support of the disclosed intraoperative approach, PARPi-fl accumulation was shown in whole excised xenografts. PARPi-fl accumulation can also be detected using a fluorescence endoscope and a custom built dual-axis confocal microscope, in the form of a large or handheld or portable device.

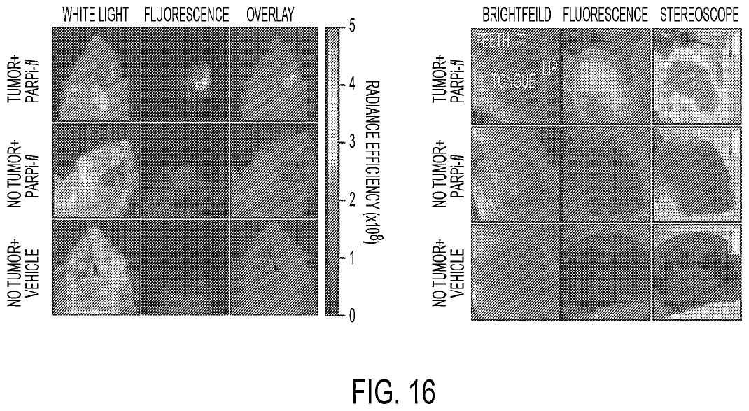

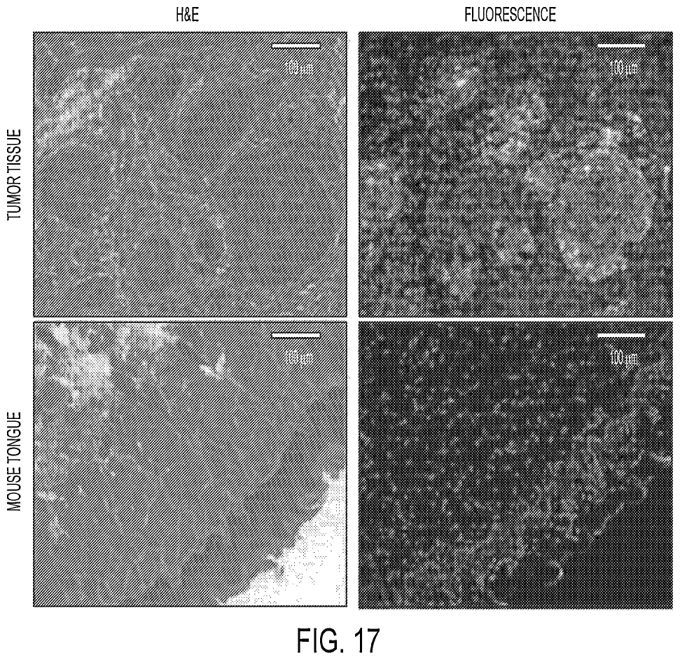

Next, the imaging approach was tested in an orthotopic tongue tumor model using FaDu cells. This approached confirmed that sufficiently high PARPi-fl accumulation compared to the surrounding healthy tissue was able to be detected in tongue tumors. As shown in FIG. 16, epifluorescence imaging and fluorescence stereoscopic imaging were used to detect PARPi-fl accumulation in tongue tumors compared to healthy tissue. Using both imaging methods revealed high accumulation of PARPi-fl in tongues with tumors and no accumulation of PARPi-fl in tongues of healthy animals that had been injected with PARPi-fl. Furthermore, the accumulation was confined to areas that were macroscopically identifiable as pathologic. These areas yielded a high contrast to the mouth cavity, the teeth, and the skin. Histological evaluation of sections of tumor bearing tongues confirmed that PARPi-fl is only found in regions that were classified as tumor tissue in H&E stained sections as shown in FIG. 17.

The above examples have shown in models of cancer that PARPi-fl, a fluorescent PARP1-targeted small molecule, specifically binds to PARP1 with a similar affinity to olaparib (Lynparza, Astra-Zeneca), an FDA-approved PARP1 inhibitor. These studies were performed in cell culture and with ex vivo imaging of excised tumors.

### Example 2: Detection and Delineation of Oral Cancer with a PARP1 Targeted Optical Imaging Agent

### Detection of PARP1 in Tumors of Human Oral Specimen

The expression of PARP1 in human OSCC was first determined using biospecimens from OSCC patients and identified mouse models of OSCC that reflect the human disease, including the expression levels of PARP1. PARPi-fl in these mouse models was tested and confirmed to be clinically relevant, non-invasive imaging systems that are capable of visualizing OSCC with high contrast after both intravenous and topical administration of PARPi FL. Without wishing to be bound to any theory, this suggested that PARPi-fl can be used to answer diagnostically relevant questions in the clinic.

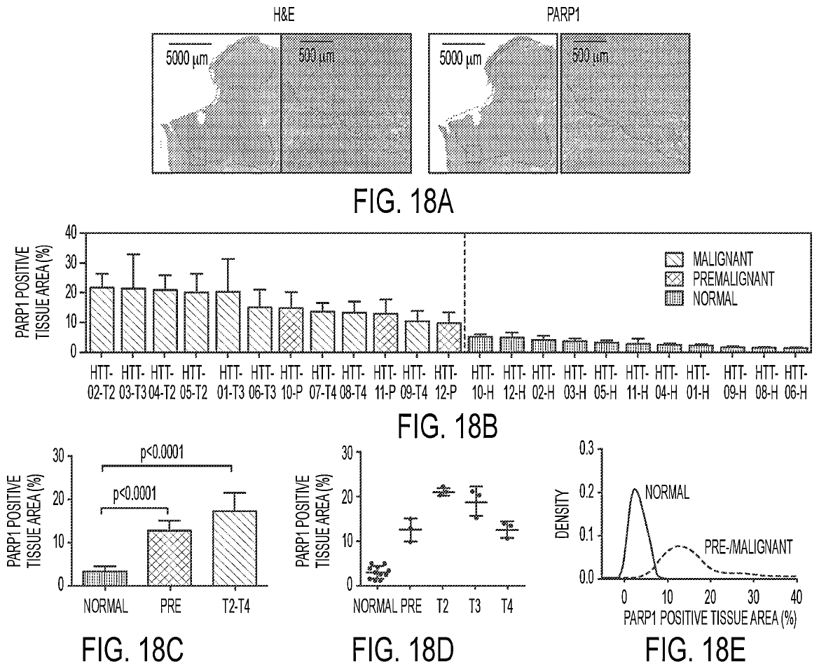

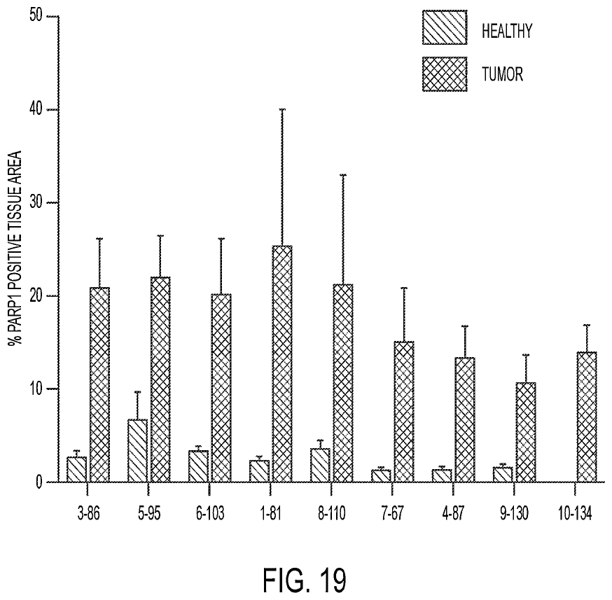

To demonstrate that high expression of PARP1 exists in human oral cancer specimen, human tongue tumor biopsies for PARP1 were used for Immunohistochemistry (n=10, 3 specimens each in tumor stage T2, T3 and T4). It was found that there is a distinction between PARP1 expression in malignant tissue compared to adjacent normal tissue areas. Quantification of the PARP1 positive area showed that in the mean of all human samples, 18.0±4.9% of the tumor area is PARP1 positive, whereas in healthy tongue tissue only 2.9±1.9% of the tissue area shows PARP1 expression (p<0.0001, students t-test). Looking at individual samples, the PARP1 positive area is markedly higher in each sample as shown in FIGS. 18A-18E and FIG. 19.

PARP1 Expression in Human Oral Cancer Biospecimens

To determine the relevance of PARP1 as a biomarker for OSCC, PARP1 expression patterns in human oral cancer tissues, along with PARP1 expression in adjacent healthy tissues in 12 human tongue tumor specimens were obtained from the Department of Pathology at Memorial Sloan Kettering Cancer Center (MSK), which were histopathologically staged using H&E stained biopsy tissue following the standard tumor, node, metastasis (TNM) classification.

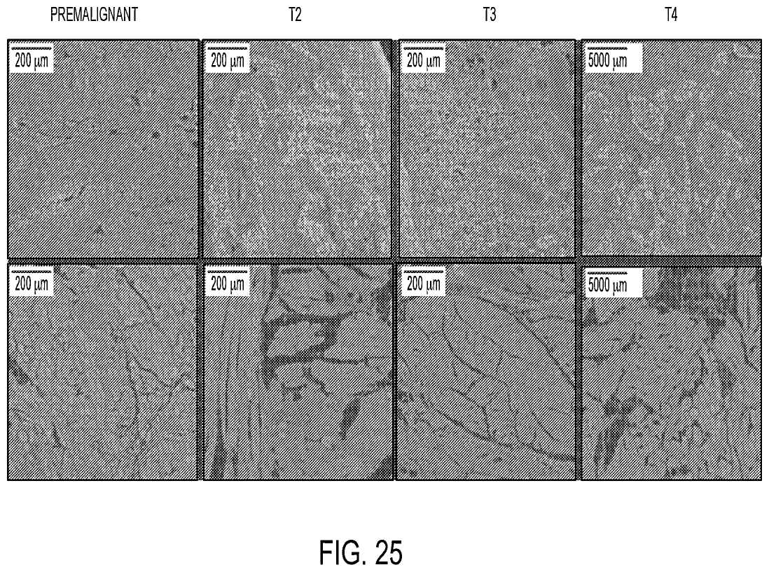

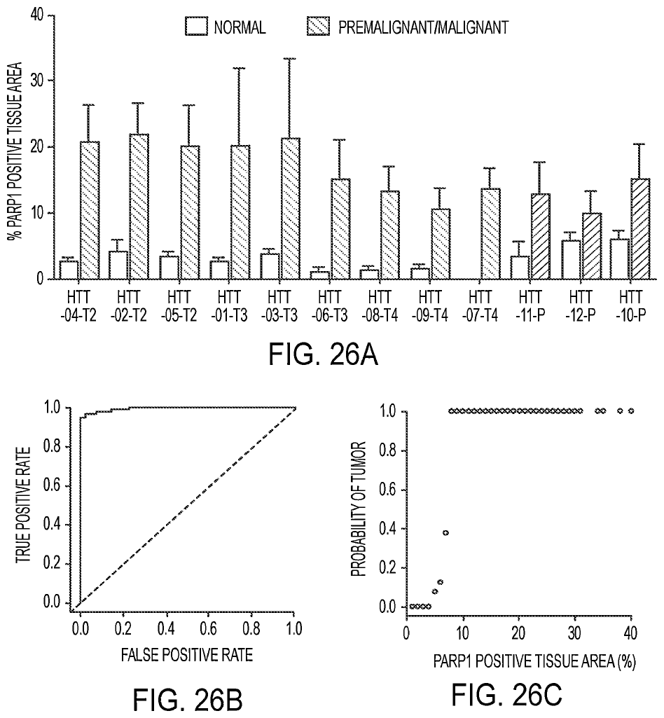

The tissues included three specimens per tumor stage: premalignant, T2, T3, and T4 (Table 1). The premalignant tissues were classified as moderate/severe dysplasia and squamous cell carcinoma in situ. The three specimens per tumor stage except for one were obtained from the edges of the tumors and featured both tumor tissue as well as healthy surrounding tissue (FIG. 18A). Strong PARP1 expression as determined by immunohistochemistry (IHC) clearly distinguished tumor from adjacent normal tissue (FIGS. 18A-18E and FIG. 25). In normal tissue, the PARP1-positive area on IHC ranged from about 1.2% to 5.3%, whereas for all malignant and premalignant specimens, PARP1-positive area on IHC ranged from about 9.7% and 21.2%, with no significant overlap between the two groups (FIG. 18B and FIG. 26A). The mean PARP1-positive area was 3.1±1.4% in normal tissue, 12.6±2.5% (P<0.0001 vs. normal) in premalignant tissue, and 17.4±4.2% (P<0.0001 vs. normal, FIG. 18C) in malignant tissue.

TABLE 1

| | | |
|---|---|---|
| Characteristic | No. of patients | Percent (%) |

Age at diagnosis

| | | |
|---|---|---|
| Median (range) | 63.2 (34.6-78.8) | — |

Sex

| | | |
|---|---|---|
| Male | 6 | 50 |
| Female | 6 | 50 |

Tumor stage

| | | |
|---|---|---|
| Premalignant | 3 | 25 |
| T2 | 3 | 25 |
| T3 | 3 | 25 |
| T4 | 3 | 5 |

Nodal Status

| | | |
|---|---|---|
| N0 | 4 | 33 |
| N1 | 2 | 17 |
| N2a | 0 | 0 |
| N2b | 6 | 50 |

Differentiation

| | | |
|---|---|---|
| Well | 2 | 17 |
| Moderate to well | 2 | 17 |
| Moderate | 5 | 42 |
| Poorly | 1 | 8 |
| Unknown | 1 | 8 |

Surgery (first-line therapy)

| | | |
|---|---|---|
| No | 0 | 0 |
| Yes | 12 | 100 |

Differences in PARP1 expression was also observed when comparing different tumor stages, albeit these differences in PARP1 expression are based on a small sample size (n=3 per tumor stage) (FIG. 18D). T2-staged tumors featured the highest PARP1 expression (21.9±1.0%), followed by T3-staged specimens (18.9±3.3%), T4-staged tumors (12.6±1.8%), and premalignant tissues (12.5±2.6%). Pooling all PARP1 expression values of malignant and normal specimens (5 to 10 values per specimen) in a density plot shows that the overlap between malignant and normal populations was very small (FIG. 18E). The variation of the PARP1-positive area values in normal tissue resulted in a narrow symmetric density plot between 0 and 10% PARP1-positive area, whereas malignant tissue showed a broader distribution. However, the distribution was asymmetric with very few data points below 10% PARP1-positive area and a long tail with up to 40% PARP1-positive tissue area (FIG. 18E).

The performance of PARP1 as a classifier for tumor and normal tissue was evaluated using a receiver operating characteristic (ROC) curve (FIG. 26B). For all samples, tumor tissue was detected by PARP1 expression with a specificity of 0.972 and a sensitivity of 0.974. The positive predictive value (PPV) was 0.982 and the negative predictive value (NPV) was 0.958 for correct classification of tumor and normal tissue, when the threshold between malignant and normal was set at 6.5% PARP1-positive area. The probability of a tissue sample was further determined to be malignant based on its PARP1-positive area and found that the probability of a given tissue area to be tumor increased from 0% to 100% between 5% and 9% PARP1-positive area (FIG. 26C).

### PARP1 Protein Expression in OSCC Xenografts

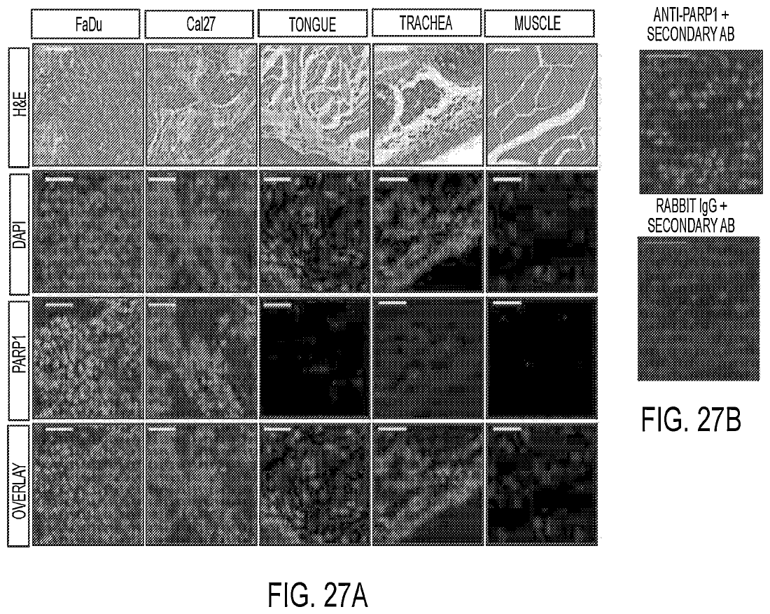

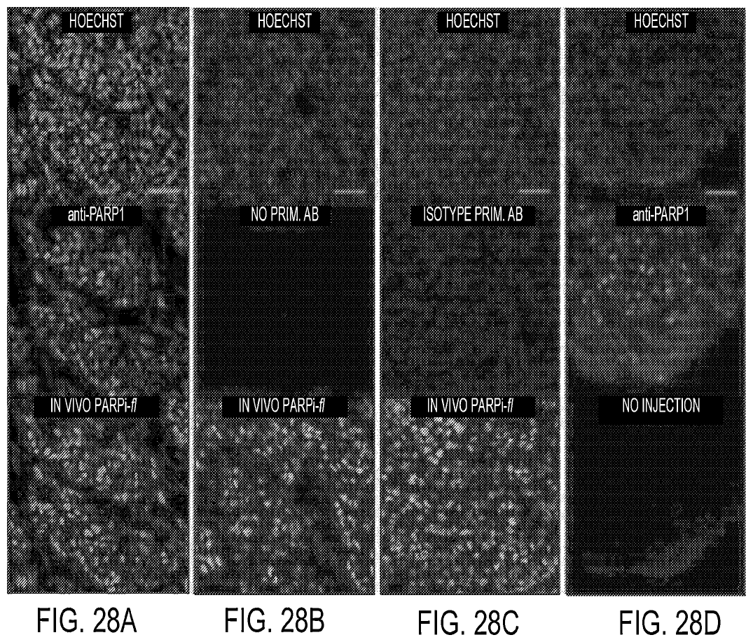

Expression of PARP1 was found to be similar in two xenograft models of human OSCC (FIGS. 18A-18B). The two OSCC cell lines FaDu and Ca127 contained PARP1 in 19.9±6.4% (FaDu) and 17.4±5.5% (Ca127) of the tissue area and displayed a PARP1 immunofluorescence intensity of 34.1±7.6 AU (FaDu) versus 19.1±5.5 AU (Ca127), respectively. These values were highly elevated compared to normal mouse tongue, trachea, and muscle, in which the PARP1-positive area was below 2% and the maximum intensity was 1.2 AU and 3.2 AU for muscle and tongue, respectively (FIG. 15C). PARP1 immunofluorescence staining was only positive in the nuclei of tumor cells, but not stromal cells or cytoplasmic areas of cancer cells (FIG. 27A), as described previously. Furthermore, negligible non-specific staining was observed when the anti-PARP1 antibody was replaced with a non-specific rabbit IgG isotype control (FIG. 27B).

Ex Vivo PARP1 Imaging with PARPi-fl in Subcutaneous OSCC Xenografts

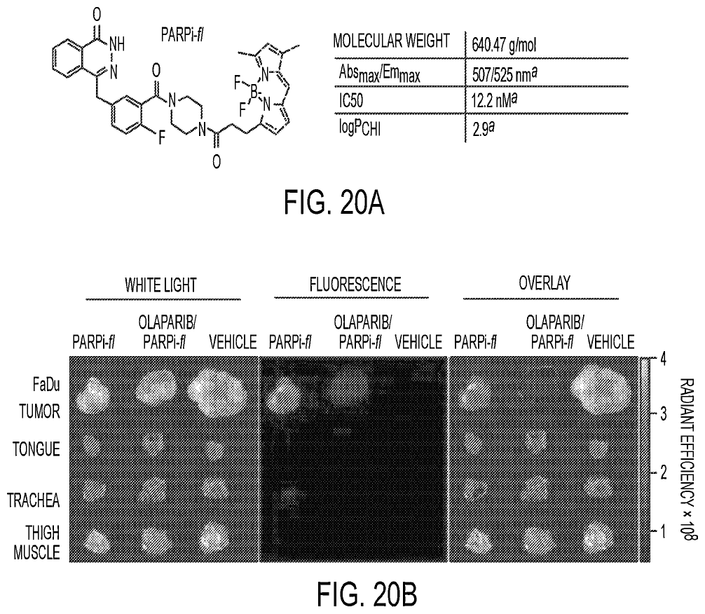

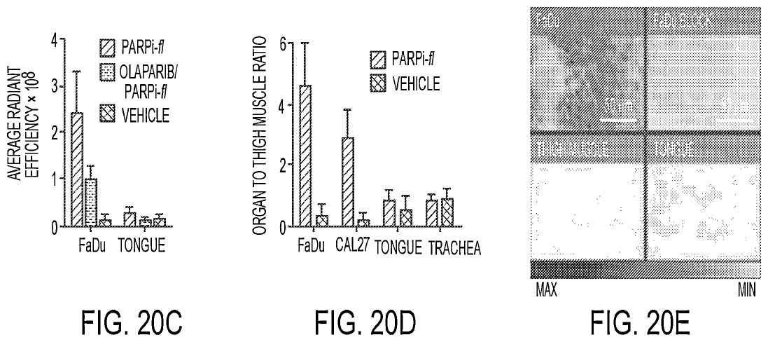

Next, it was determined whether FaDu and Ca127 tumors accumulated PARPi-fl after intravenous injection, and if tumor uptake was due to binding to PARP1. PARPi-fl is a targeted imaging agent that fluoresces in the visible range (FIG. 20A) and accumulates in the nuclei of PARP1-expressing cells. Epifluorescence imaging of excised subcutaneous FaDu and Ca127 tumors was performed 90 minutes after injection of PARPi-fl (75 nmol PARPi-fl, 0.5 mM, in PBS with 30% PEG300), and the intensity of the fluorescence signal was compared to normal tongue, trachea, and control thigh muscle tissue. PARPi-fl generated a strong fluorescence signal in tumors and almost no fluorescence in normal tissues (FIG. 20B). The specificity of the signal was confirmed by injecting the non-fluorescent PARP1-targeted drug olaparib before administration of PARPi-fl, which resulted in a reduction of the fluorescent signal of the tumor by 60% (average radiant efficiency PARPi-fl: 2.4×10⁸ versus olaparib/PARPi-fl: 0.98×10⁸, P<0.001, FIG. 20C). The signal in the tongue was low, independent of the blocking (average radiant efficiency PARPi-fl: 0.24×10⁸ versus olaparib/PARPi-fl: 0.13×10⁸, P=0.3, FIG. 20C), further supporting that there is little expression of PARP1 in tongue tissue, and that little of the imaging agent is nonspecifically bound. In control mice injected with vehicle only, fluorescence signals did not exceed an average radiant efficiency of 0.11×10⁸ in either tumor or tongue.

The fluorescence signal was quantitatively evaluated by tissue-to-thigh-muscle ratios. This ratio was 4.6±1.4 for FaDu tumors and 2.9±1.0 for Ca127 tumors (FIG. 20D). In mouse tongues and trachea, which represent the surrounding healthy tissues in oral cancer, the fluorescence signals were not elevated compared to thigh muscle (uptake ratio: 0.8±0.3 for tongue, and 0.3±0.2 for trachea). Microscopic analysis of the fluorescence distribution in freshly excised tissue further confirmed the specific accumulation of PARPi-fl in tumor cells, since a strong nuclear fluorescence was only observed in FaDu tumors, but not in tongue or muscle after PARPi-fl injection (FIG. 20E).

Following IV injection in mice, PARPi-fl was cleared rapidly from the circulation with an a half-life of 1.2 min and a β half-life of 88 min. PARPi-fl was rapidly taken up by cancer cells in tumor xenografts and reaches the nucleus within minutes (FIGS. 20B-20C) where it remained bound for several hours while the fluorescence is cleared from the cell membrane and cytosol (FIGS. 20B-20C). The tumor uptake by xenografts is due to PARP1 binding as it can be almost completely inhibited by coinjection Olapirib. PARPi-fl is metabolically stable in mice with less than 50% metabolites at 30 min post injection, the time of peak uptake of PARPi-fl in subcutaneous tumors.

### Correlation of PARP1 Expression on IHC and PARPi-fl Localization

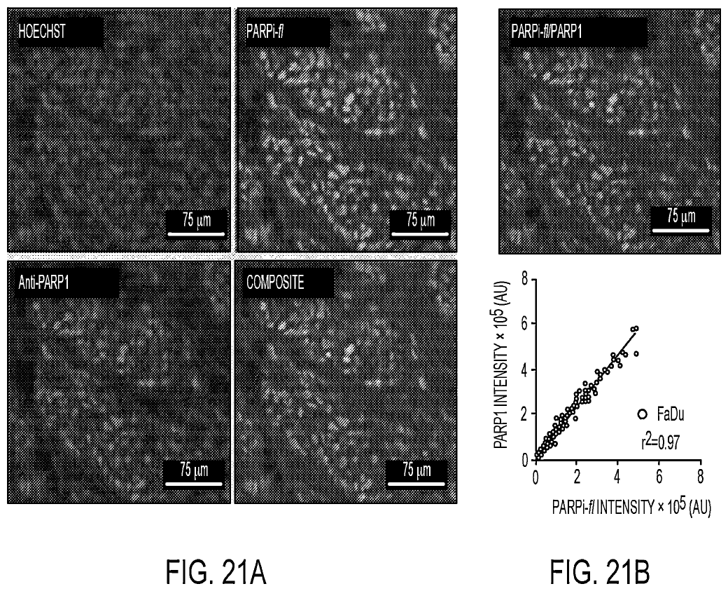

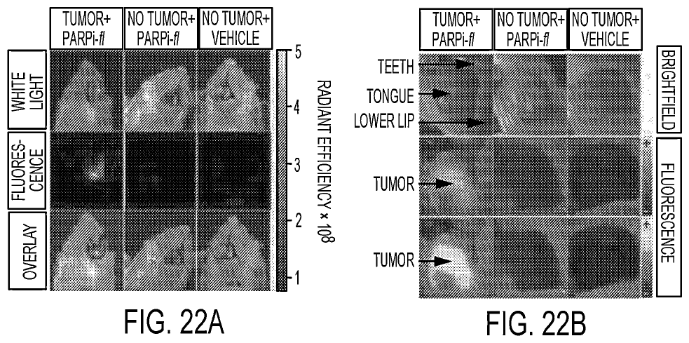

PARP1 antibody staining was highly co-localized with PARPi-fl fluorescence (Rcoloc.=0.986, R²=0.973; 95% confidence interval 0.98 to 0.989; FIGS. 21A and 21B). Without wishing to be bound to any theory, this data suggested that PARPi-fl not only binds specifically to PARP1-expressing cells but also quantitatively reflects the amount of PARP1 present in a cell (FIG. 21B). There was no bleed-through/cross-contamination of PARPi-fl into the PARP1 channel and vice versa, shown by PARP1 and PARPi-fl control experiments (FIGS. 28A-28D).

FIGS. 21A and 21B show localization of PARPi-fl in relation to cell nuclei and PARP1 protein using confocal imaging.

FIG. 21A shows cell nuclei were stained with Hoechst 33342 (blue), PARPi-fl was injected intravenously in vivo (green) and PARP1 was stained with an anti-PARP1 antibody and detected via Immunofluorescence (red). FIG. 21B shows correlation analysis (Pearson R squared) between intravenously injected PARPi-fl and PARP1 antibody staining in FaDu tumor tissue, based on the intensity of green or red in the nuclear area, which was determined by thresholding of the area covered by blue fluorescence (Hoechst). 222 xy pairs pooled from n=3 tumor sections.

### PARP1-fl Optical Imaging of Orthotopic OSCC

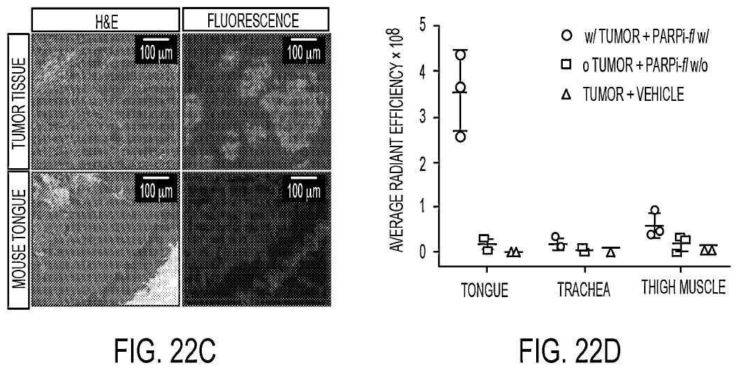

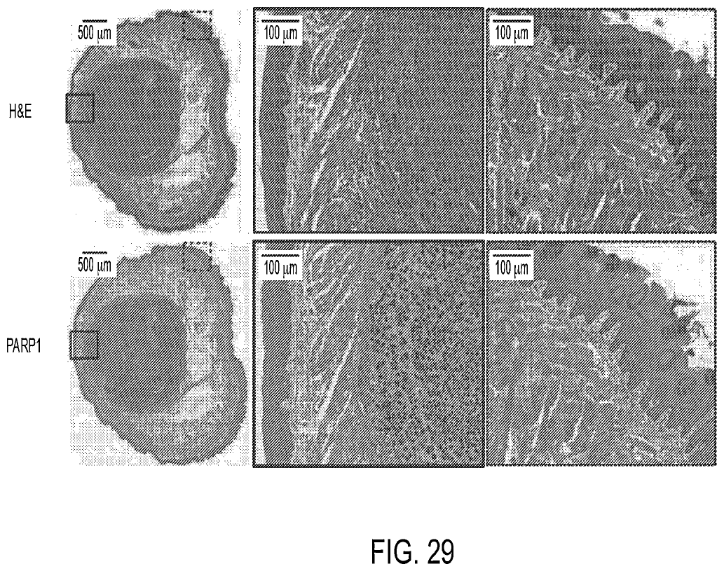

PARPi-fl uptake was also imaged in vivo in an orthotopic tongue tumor model of OSCC (FaDu cells) using the same parameters as for subcutaneous tumor imaging ex vivo (intravenous injection of 75 nmol PARPi-fl/animal, imaging 90 minutes post-injection). Here, epifluorescence imaging showed a strong PARPi-fl accumulation in parts of the tongue that were visibly affected by OSCC, whereas no signal accumulation was observed in tongues without tumors after PARPi-fl or vehicle injection (FIG. 22A). Further, PARPi-fl accumulation in tongue tumors was visualized using a fluorescence stereoscope. This imaging technique is closer to the clinical situation, where real-time fluorescence imaging is required. PARPi-fl accumulation in orthotopic tumors was clearly visible and confined to areas that were macroscopically identifiable as pathologic (FIG. 22B). Histological evaluation of tissue sections from tumor-bearing tongues confirmed that PARPi-fl was only be found in regions classified as tumor tissue in H&E stained sections (FIG. 22C), and that the tumors showed highly elevated PARP1 expression compared to normal mouse tongue (FIG. 29). Ex vivo signal quantification in excised orthotopic and healthy tongues, trachea, and muscle revealed a 6.1-fold higher radiant efficiency in orthotopic tumors than in thigh muscle (3.5±0.9×10⁸ and 0.6±0.3×10⁸ for orthotopic tumors and thigh muscle, respectively; P<0.01). When no tumor was present tongue and thigh muscle showed the same fluorescence signal (radiant efficiency 0.2±0.1×10⁸ and 0.2±0.2×10⁸, respectively; P=0.1) (FIG. 22D).

### Cellular Resolution Imaging of PARP1-fl in Whole Tumors

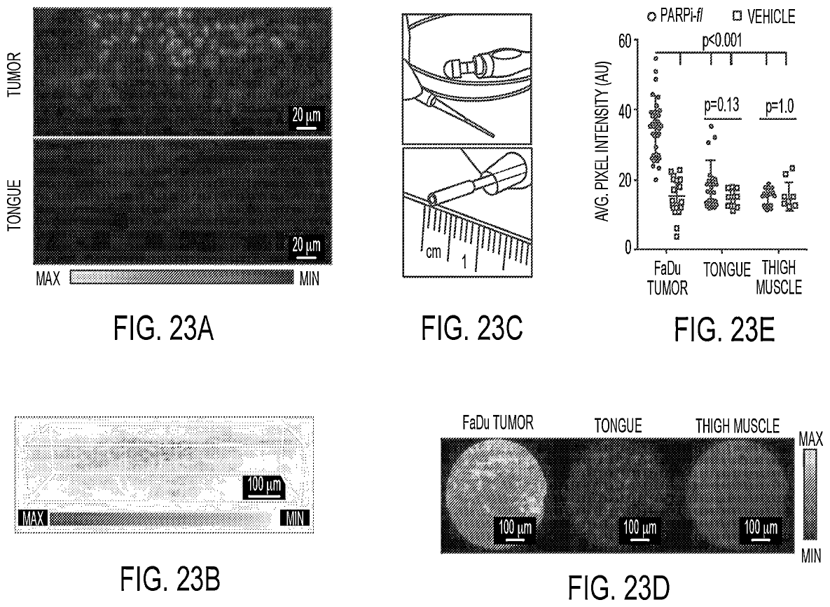

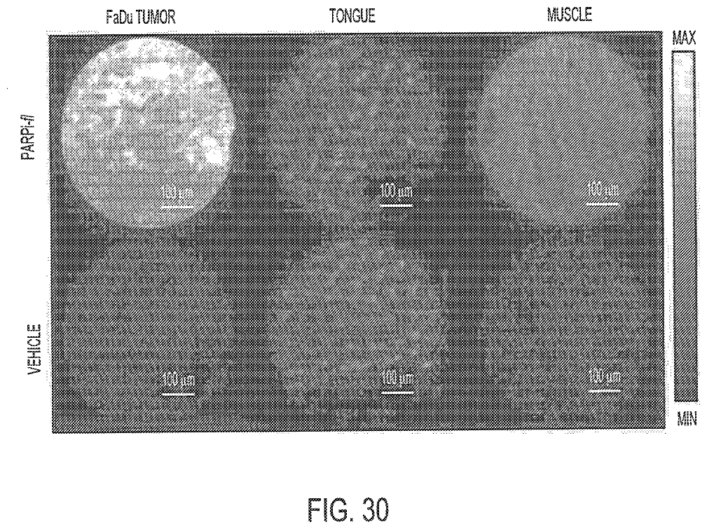

To show that PARPi-fl is suitable for imaging of tumors at cellular resolution, freshly excised FaDu tumor tissue were imaged 90 minutes after injection of PARPi-fl using a custom dual-axis confocal microscope at a range of depths (FIG. 3A and FIGS. 23A and 23B) and a commercial fluorescence endomicroscope at a fixed depth (FIGS. 23C and 23D). This instrument enabled the identification of single cells based on their nuclear PARPi-fl uptake at up to 200 μm below the tissue surface, whereas negligible signal was detected in normal tongue tissue from animals injected with PARPi-fl (FIG. 23B and FIG. 3B). The dual axis confocal microscopy technique can be developed into a hand-held device, which can then be translated into clinical practice in the future. Fluorescence endomicroscopy, a technique that has already been implemented in clinical practice, was also able to clearly distinguish between FaDu tumors and control tissues (FIG. 23C). When no PARPi-fl was injected, no difference in fluorescence intensity of FaDu tumors, tongue, or muscle tissue was observed (FIG. 30). When compared to the vehicle control group, the average signal intensity of FaDu tumors was significantly increased 90 minutes after PARPi-fl injection (35.4±8.6 AU and 15.2±5.0 AU, respectively; P<0.001). There was no difference between the average signal intensity after PARPi-fl or vehicle injection in tongue and thigh muscle (18.5±6.9 AU and 15.0±2.5 AU for tongue, with and without PARPi-fl administration, respectively, P=0.13; 15.1±2.4 AU and 15.1±4.0 AU for thigh muscle, with and without PARPi-fl administration, respectively, P=1.0; FIG. 23E).

Oral Cancer Imaging after Topical Application of PARPi-fl

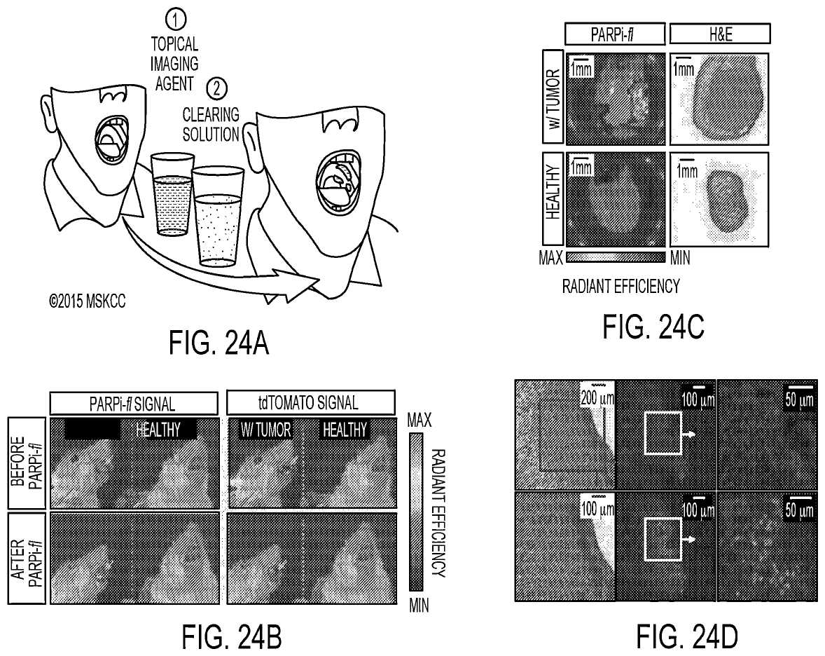

Topical application of a PARPi-fl formulation (30% PEG300/PBS) with subsequent fluorescence screening of the oral cavity for OSCC detection can improve the current standard of care, particularly in low resource settings. For example, FIG. 24A shows a schematic of an exemplary imaging procedure using PARPi-fl. PARPi-fl can be administered locally followed by rinsing with a clinical solution. Patients can then undergo fluorescence imaging of the oral cavity. The location of the tumor can be documented by photographic imaging and the intensity of fluorescence in the tumor region relative to adjacent normal mucosa can be quantified.

In a preclinical model of tongue OSCC, it was investigated if PARPi-fl tumor contrast after topical application was comparable to intravenous injection. Macroscopic evaluation using epifluorescence imaging confirmed that PARPi-fl colocalized to areas of the tongue where there was the orthotopic tumor, as confirmed by tumor cells expressing the fluorescent protein tdTomato (FIG. 24B). Correlation of PARPi-fl signal with H&E staining of tongue sections confirmed that the agent's retention after topical application is strongest in regions of tumor growth, but only if it is close to the surface of tongue (FIG. 24C). Microscopic evaluation revealed that PARPi-fl was able to penetrate up to 300 μm deep into tumor tissue during the one minute application window, whilst being able to wash out from non-target areas during the cleaning steps, resulting in high contrast PARP1 staining of OSCC nuclei (FIG. 24D).

These results indicate that PARP1 protein expression is markedly increased in OSCC when compared to normal tissues of the oral cavity. Moreover, it was demonstrated that the small molecular imaging agent PARPi-fl can be used to delineate OSCC in living mice. PARPi-fl is efficiently retained in oral cancer tissue, yielding a strong imaging signal, paired with high contrast to surrounding normal tissue. This enabled high-resolution in vivo imaging of orthotopic OSCC with clinically translatable instruments. Using these devices, PARP1 expression was imaged from the macroscopic to the subcellular level. It was also shown that PARPi-fl, due to its high tissue permeability (4.7±2.5 μm/s), efficiently penetrates into tumor tissue after topical application, and selectively accumulates in OSCC cells close to the tissue surface, while being washed out from non-target tissues and compartments within minutes.

PARP1 expression was elevated throughout the patient-derived OSCC samples. PARP1 expression per nucleus was fairly uniform. However, the density of PARP1-positive tumor cells varies in different areas. Specifically, PARP1 expression levels were higher at the invasive margins of the tumors than in the center. The impact of tumor cell density is also apparent in FIG. 18D, where T2 specimens have higher PARP1 expression than the more necrotic T3 and T4 specimens. Interestingly, the premalignant specimens in the data described herein showed equally high PARP1 expression levels as malignant specimens. Premalignant tissues, such as severe dysplasia or carcinoma in situ, have been shown to be associated with progression to cancer. The overexpression of PARP1 in premalignant tumors may enable early diagnosis of OSCC and become useful in certain therapeutic applications.

PARP1 is expressed in a large number of cancers. Other members of the PARP family, such as PARP2, which is also inhibited by olaparib, is less abundant and its expression was found not to be upregulated in a number of primary cancers. Although, no data on PARP2 expression in oral cancer are currently available, this is pointing towards a less important role of PARP2 in tumorigenesis and a low suitability as cancer imaging agent compared to PARP1. Without wishing to be bound to any theory, PARP1 overexpression may be due to the increased DNA damage occurring in genetically unstable cancer cells, rather than the activation of specific oncogenic pathways. Furthermore, the density of nuclei is typically higher in malignant tumors than in most normal tissues. The PARPi-fl in vivo imaging signal therefore reflects both the higher expression levels of PARP1 per nucleus as well as the higher nuclear density in malignant tumors. Thus, PARPi-fl can be used to image a large variety of tumors during screening or surgery. OSCC is an obvious candidate for the initial evaluation of PARPi-fl imaging because of the clinical needs for better detection and delineation of OSCC, as well as its easy accessibility for fluorescence imaging.

In the field of optical fluorescence imaging, a large number of probes absorb and emit near-infrared light. In this wavelength range (650 nm-900 nm), photons are less scattered and absorbed by tissues, which allows for better tissue penetration. In addition, there is less background autofluorescence from tissues with near-infrared excitation, as compared to visible excitation. The BODIPY® FL fluorophore used to synthesize certain experimental embodiments of PARPi-fl operates in the visible range of light (400 nm-700 nm), but it has the added advantage of an exceptionally low molecular weight and it does not ionize under physiological conditions. Other fluorophores may also have these advantages. These properties allow for efficient in vivo extravasation, combined with fast cell permeation and intranuclear accumulation. Larger fluorophores and charged molecules result in significantly reduced cell permeability, together with low contrast ratios. The in vivo imaging results presented here confirm that the fluorescence from the fluorophore allows for high-contrast imaging of superficial tumors in the tongue despite the known limitations of green fluorescent dyes. Moreover, fluorescence imaging systems that operate with a green fluorescence channel have entered clinical practice, e.g., probe based confocal laser endomicroscopy (pCLE), which is FDA-approved for imaging the entire gastrointestinal tract, including the oral cavity. The utility of pCLE imaging for better differentiation of nondysplastic, precancerous, and cancerous lesions of the head and neck in patients has already been shown using fluorescein, a nonspecific green fluorescent dye, which absorbs and emits very closely to PARPi-fl (Excitation/Emission max.; fluorescein: 490/525 nm; PARPi-fl: 507/525 nm). PARPi-fl accumulation in OSCC was imaged with high contrast using a clinically approved pCLE system.

Using a PARPi-fl assisted oral cancer screening procedure, the application method of PARPi-fl can be switched from intravenous to topical application, reducing complexity, and increasing the agent's breadth and versatility in the clinic. Topical application further reduces cost and potential side effects, and streamlines the imaging protocol. Optical fluorescence imaging equipment is lower priced and has a higher grade of mobility compared to other molecular imaging modalities, for example PET or MM.

PARPi-fl was shown to penetrate up to 300 μm into tissue, which is sufficient for detection of OSCC, a disease that typically originates within the outermost cell layers of the oral cavity. In conclusion, the results described herein indicate that PARPi-fl imaging of OSCC is very promising for a variety of applications, including cancer screening, surgical guidance during tumor removal, and delineation of tumor margins by pCLE. Hence, PARP1 imaging can result in earlier detection of oral cancer and reduce the morbidity of radical surgery that plagues patients suffering from OSCC.

### Toxicity

The toxicity of several BODIPY-FL labeled molecular imaging agents has been evaluated in cell culture studies. Toxic effects were only observed after prolonged exposure at concentrations of 10 μM or more. The toxicity of PARPi-fl was compared with Olapirib in two glioblastoma cell lines (e.g., U87 and U251). The IC50s in an MTT assay for olaparib and PARPi-fl were 28 μM and 24 μM in U87 cells, and 8.0 and 5.5 in U251 cells, indicating that in these two cell lines PARPi-fl was not more toxic than olaparib.

Local administration of 20 μl of a 400 μM solution of PARPi-fl was tested on the oral mucosa of mice. This dose caused no local irritation, and no changes in clinical chemical and hematologic parameters (Tables 2A and 2B).

Tables 2A (Clinical chemistry) and 2B (Hematology) shows toxicity of PARPi-fl after local administration on the oral mucosa of mice. Cohorts of mice (6-8 weeks old female athymic mice) were administered a solution of PARPi-fl as a topical application (29 nmol PARPi-fl in 50 μL), and incubated for 10 min, before excess agent was washed off. Mice received blood draws after 24 h and 48 h post administration, and were then sacrificed to receive a full necropsy.

TABLE 2A
Clinical chemistry

| | Unit | Ref range | PARPi-fl |
|---|---|---|---|
| ALP | IU/L | 23-181 | 90 ± 18 |
| ALT (SGPT) | IU/L | 16-58 | 31 ± 6 |
| AST (SGOT) | IU/L | 36-102 | 67 ± 4 |
| GGT | IU/L | 0-2 | 0 |
| Albumin | g/dl | 2.5-3.9 | 2.7 ± 0.2 |
| Total Protein | g/dL | 4.1-6.4 | 4.9 ± 0.8 |
| Globulin | mg/dl | 1.3-2.8 | 2.3 ± 0.7 |
| Total Bilirubin | mg/dl | 0.0-0.3 | 0.1 ± 0.0 |
| Creatinine | mg/dl | 0.1-0.6 | 0.24 ± 0.01 |
| Cholesterol | mg/dl | 74-190 | 86 ± 8 |
| BUN | mg/dl | 14-32 | 28 ± 2 |
| Glucose | mg/dl | 76-222 | 229 ± 54 |
| Calcium | mg/dl | 7.6-10.7 | 9.6 ± 0.2 |
| Phosphorus | mg/dl | 4.6-9.3 | 6 ± 1 |
| Chloride | mEq/L | 103-115 | 114 ± 1 |
| Potassium | mEq/L | 3.4-5.5 | 3.8 ± 0.7 |
| Sodium | mEq/L | 146-155 | 151 ± 3 |
| A/G ratio | — | 1.0-2.2 | 1.2 ± 0.3 |
| Na/K ratio | — | 26.7-42.1 | 41 ± 7 |

TABLE 2B
Hematology

| | Unit | Ref range | PARPi-fl |
|---|---|---|---|
| WBC | K/μL | 1.8-10.7 | 5.78 ± 0.90 |
| NEUTROPHI | K/μL | 0.1-2.4 | 1.69 ± 0.04 |
| LYMPHOCYT | K/μL | 0.9-9.3 | 3.34 ± 1.07 |
| MONOCYTE | K/μL | 0.0-0.4 | 0.10 ± 0.04 |
| EOSINOPHIL | K/μL | 0.0-0.2 | 0.64 ± 0.55 |
| BASOPHILS | K/μL | 0.0-0.2 | 0.01 ± 0.02 |
| NE % | % | 6.6-38.9 | 29.5 ± 4.0 |
| LY % | % | 55.8-91.6 | 57.0 ± 10.9 |
| MO % | % | 0.0-7.5 | 1.9 ± 0.8 |
| EO % | % | 0.0-3.9 | 5.5 ± 1.0 |
| BA % | % | 0.0-2.0 | 0.2 ± 0.3 |
| RBC | M/μL | 6.36-9.42 | 10.24 ± 0.62 |
| HB | g/dL | 11.0-15.1 | 15.1 ± 0.9 |
| HCT | % | 35.1-45.4 | 50.4 ± 2.9 |
| MCV | fL | 45.4-60.3 | 49.3 ± 1.2 |
| MCH | Pg | 14.1-19.3 | 14.7 ± 0.4 |
| MCHC | g/dL | 30.2-34.2 | 30.0 ± 0.1 |
| RDW | % | 12.4-27.0 | 23.4 ± 0.6 |
| PLT | K/μL | 592-2972 | 1077 ± 171 |
| MPV | fL | 5.0-20.0 | 6.1 ± 0.2 |

### Clinical Data, Pharmacology, and Toxicology

There is no clinical experience with PARPi-fl or BODIPY-FL. However, there are data on the FDA approved PARP1 inhibitor olaparib which is the PARP1 binding motif of PARPi-fl. These are briefly summarized in the following paragraphs.

The approved dose of olaparib for treatment of ovarian cancer is 400 mg orally twice daily. Treatment is typically given continuously over several months. Following oral administration olaparib is rapidly absorbed with peak plasma concentrations between 1-3 hours after dosing. The apparent volume of distribution is more than 150 L, indicating intracellular accumulation. olaparib is metabolized via CYP34A and the metabolites are excreted via urine and bile. The terminal half-life is 11.9 hours after administration of a 400 mg dose. More than 86% of ¹⁴C-labeled olaparib was excreted within 7 days.

At the approved dose and dose schedule olaparib is well tolerated. Reported side effects in patients with advanced ovarian cancer being treated with olaparib include anemia, abdominal pain, decreased appetite, nausea, vomiting, diarrhea, dyspepsia and pharyngitis.

### Materials and Methods

### Cell Culture

The OSCC cell lines FaDu (hypopharyngeal SCC; ATCC, Manassas, Va.) and Ca127 (tongue SCC; ATCC, Manassas, Va.) were grown in a monolayer culture at 37° C. in a 5% CO2 humidified atmosphere. FaDu cells were maintained in MEM medium and Ca127 cells in D-MEM medium, both containing 10% (v/v) FBS and 1% PenStrep.

### Animal Models

Female athymic nude mice (NCr-Foxn1nu, Taconic, Hudson, N.Y.) were housed under standard conditions with water and food ad libitum. Throughout all procedures, animals were anesthetized with 2% isoflurane. To implement subcutaneous human OSCC tumors, 2×10⁶ FaDu or Ca127 cells were dispensed in 100 μl of a 1/1 mixture of medium/Matrigel™ (BD Biosciences, Bedford, Mass.) and were injected into the lower back of the animals. Experiments were conducted when tumors reached 100-150 mm³ volume. For an orthotopic OSCC model, 5×10⁵ FaDu or FaDu_{tdTomato} (FaDu stably transfected with tdTomato fluorescent protein; Creative Biogene, Shirley, N.Y.) cells in 20 μl PBS were injected directly into the tongue and the mice were observed daily for tumor growth and weight loss. Imaging was conducted usually after 3-4 weeks. All animal experiments were performed in accordance with institutional guidelines and approved by the IACUC of MSK, and followed NIH guidelines for animal welfare.

### Human Tissues

Quantification of PARP1 expression was carried out using human tongue tumor specimens (n=12), obtained from the Department of Pathology of MSK. The use of tissues was approved by the Institutional Review Board (IRB) at MSK and informed consent was obtained from all subjects.

### PARP1 Expression in Tissues

PARP1 antigen in human oral cancer tissue, as well as FaDu and Ca127 xenografts and mouse tissues was detected using immunohistochemical (IHC) and immunofluorescence (IF) staining techniques, which were performed at the Molecular Cytology Core Facility of MSK using the Discovery XT processor (Ventana Medical Systems, Tucson, Ariz.). The anti-PARP1 rabbit polyclonal antibody (sc-7150, Santa Cruz Biotechnology, Santa Cruz, Calif.) specifically bound both human and mouse PARP1 (0.2 μg/ml). Paraffin-embedded formalin fixed 3 μm sections were deparaffinized with EZPrep buffer, antigen retrieval was performed with CC1 buffer (both Ventana Medical Systems, Tucson, Ariz.), and sections were blocked for 30 minutes with Background Buster solution (Innovex, Richmond, Calif.). Anti-PARP1 antibody was incubated for 5 hours, followed by 1 hour of incubation with biotinylated goat anti-rabbit IgG (PK6106, Vector Labs, Burlingame, Calif.) at a 1:200 dilution. For IHC detection, a DAB detection kit (Ventana Medical Systems, Tucson, Ariz.) was used according to the manufacturer's instructions, sections were counterstained with hematoxylin and coverslipped with Permount (Fisher Scientific, Pittsburgh, Pa.). IF detection was performed with Streptavidin-HRP D (from DABMap Kit, Ventana Medical Systems), followed by incubation with Tyramide Alexa Fluor 594 (T20935, Invitrogen, Carlsbad, Calif.) prepared according to the manufacturer's instructions. Sections were counterstained with 4′,6-diamidino-2-phenylindole (DAPI) for 10 minutes and coverslipped with Mowiol® mounting medium (Sigma-Aldrich, St. Louis, Mo.). Incubating with a rabbit IgG instead of the primary antibody controlled for non-specific binding of the secondary antibody. For morphological evaluation of tissue characteristics, H&E staining was performed on adjacent sections.

### Quantification of PARP1 Expression

For PARP1 protein quantification, stained tumor sections were digitalized using a MIRAX Slide Scanner (3DHISTECH, Budapest, Hungary). On at least 10 fields of view per section, PARP1 presence was quantified using MetaMorph® Software (Molecular Devices, Sunnyvale, Calif.). In IHC stained tissues, a thresholding was performed on brown (PARP1) and blue (tissue) areas and the relative PARP1-positive area was calculated by dividing the brown area by the total tissue area. For IF, the PARP1-positive area was determined by thresholding the red fluorescent area and dividing it by the whole tissue area, which was determined based on autofluorescence in the green channel. PARP1 intensity was also determined by measuring the red fluorescence intensity in all nuclei, which were thresholded using DAPI staining. The measured fluorescence intensities were averaged over all nuclei in each field of view, with intensity values ranging from 0-255.

### Synthesis of PARP1-fl

Synthesis of the optical imaging agent PARPi-fl was carried out as described herein. The green fluorescent dye BODIPY-FL NETS-ester (Invitrogen, Carlsbad, Calif.) was conjugated to 4-(4-fluoro-3-(piperazine-1-carbonyl)benzyl)phthalazin-1(2H)-one and purification by preparative HPLC (Waters' XTerra C-18 5 μm column, 7 ml/min, 5% to 95% of acetonitrile in 15 min) afforded PARPi-fl in 70-79% yield as a red solid. Analytical HPLC analysis (Waters' Atlantis® T3 C18 5 μm 4.6×250 mm column) showed high purity (>97%) of the imaging agent. The identity of PARPi-fl was confirmed using ESI-MS (MS(+) m/z=663.4 [M+Na]+). For imaging studies, PBS (117 μl) was slowly added to an aliquot of PARPi-fl (50 μg, 75 nmol) in 50 μl of poly(ethylene glycol) (PEG300, Sigma-Aldrich, St. Louis, Mo.) to obtain a final injection volume of 167 μl.

### Imaging of PARP1-fl Uptake in Subcutaneous Human OSCC Xenografts

For evaluation of the uptake of PARPi-fl in subcutaneous OSCC xenografts, animals carrying either FaDu or Ca127 tumors were intravenously injected with PARPi-fl (75 nmol/167 μl PBS with 30% PEG300 (Sigma-Aldrich, St. Louis, Mo.)) (n≥6/group). To assess the specificity of PARPi-fl accumulation in one group of animals, a 50-fold excess (3.75 μmol/100 μL PBS with 30% PEG300) of olaparib (LC Laboratories, Woburn, Mass.) was injected 30 minutes prior to the PARPi-fl injection, blocking the specific binding sites in FaDu tumors (n=4). Animals were sacrificed 90 minutes post-injection and tumors, tongues, trachea, and muscle were excised and imaged using epifluorescence imaging (IVIS Spectrum, PerkinElmer, Waltham, Mass.). For detection of the fluorescent PARPi-fl emission, a predefined GFP Filterset (excitation: 465/30 nm, emission: 520-580 nm) was used and subsequently removed autofluorescence through spectral unmixing. Semiquantitative analysis of the PARPi-fl signal was conducted by measuring the average radiant efficiency in regions of interest (ROIs) that were placed on all organs under white light guidance. This measure carries the unit [p/s/cm²/sr]/[μW/cm²] and is defined as the number of photons per second leaving a square centimeter of tissue and radiating into a solid angle of one steradian (sr). Resulting numbers are normalized for the integration time, binning, f/stop, field of view, illumination intensity, and the ROI area, making measurements comparable among each other. Freshly excised whole tumors were also microscopically imaged directly after epifluorescence imaging; tissues were placed on a cover slip with a freshly cut surface facing the cover slip and images were taken on an inverted laser scanning confocal microscope using 488 nm laser excitation (LSM 5-Live, Zeiss, Jena, Germany).

### Correlation of PARP1-fl Uptake and PARP1 Expression

To determine the specificity of the accumulation of PARPi-fl within tumor tissue, the inter- and intracellular co-localization of the targeted fluorescent probe with PARP1 antigen was determined in histological sections. FaDu xenografts and control tissues (tongue, muscle) were snap-frozen 90 minutes after intravenous injection of PARPi-fl (75 nmol/167 μl 30% PEG300 in PBS). Next, 10 μm cryosections were fixed in 4% paraformaldehyde for 8 minutes, followed by blocking with 3% (v/v) goat serum (Sigma-Aldrich, St. Louis, Mo.) in PBS. Antibodies were diluted in 1% (w/v) BSA and 0.3% (v/v) Triton X-100 in PBS. Anti-PARP1 primary antibody (sc-7150, Santa Cruz Biotechnology, Santa Cruz, Calif.) was incubated overnight at 4° C. (1 μg/ml), followed by three 10-minute washes with PBS and incubation with secondary AlexaFluor® 680 goat anti-rabbit antibody (A21076, Molecular Probes, Eugene, Oreg.) for 1 hour at 4° C. (2 μg/ml). After another 5-minute PBS wash, sections were mounted with Mowiol® (Sigma-Aldrich, St. Louis, Mo.) containing Hoechst 33342 DNA Stain (Sigma-Aldrich, St. Louis, Mo.). Fluorescence images were captured using a Leica (Buffalo Grove, Ill.) SP8-inverse confocal microscope equipped with a 405 nm laser for detection of cell nuclei, a 488 nm laser for detection of in vivo applied PARPi-fl, and a 670 nm laser for detection of PARP1 antibody stain, each paired with suitable emission filters. Incubating sections with either a nonspecific rabbit IgG or PBS instead of primary antibody confirmed binding specificity. Bleed-through of signals into other channels was excluded by imaging sections that were either not injected with PARPi-fl in vivo (no signal should be seen in the 488 nm channel) or not stained with PARP1 (no signal should be seen in the 670 nm channel). Correlation analysis between PARPi-fl and PARP1 signal intensity was performed using MetaMorph® Software (Molecular Devices, Sunnyvale, Calif.).

### Imaging of PARPi-fl Uptake in Orthotopic Human OSCC Xenografts

For orthotopic FaDu tongue tumors, epifluorescence imaging was conducted using the same procedure as described above, but animals were alive when imaged 90 minutes post-injection. All animals were anesthetized with 2% isoflurane in medical air. The tongues of all animals were exposed by opening their mouths and moving the tongue past the front teeth into the field of view of the IVIS. Animals were divided into three groups: tumor-bearing animals that were injected with PARPi-fl (75 nmol/167 μl PBS with 30% PEG300), healthy animals that were injected with PARPi-fl, and healthy animals that were injected with vehicle (167 μl PBS with 30% PEG300) (n=3/group). Afterwards, animals were sacrificed and tongues, trachea, and thigh muscle were imaged ex vivo. Using the same experimental setup, imaging with a fluorescence stereoscope was conducted to show that the PARPi-fl signal was also be detected under real-time imaging conditions, as would be the case in the clinical setting. Here, the tongues of anaesthetized animals were imaged using 500/20 nm excitation and 535/30 emission filters and a fixed exposure time of 500 ms (SteREO Lumar.V12, Zeiss, Jena, Germany). Imaging was performed 90 minutes after intravenous injection of PARPi-fl (75 nmol/167 μl PBS with 30% PEG300).

### Imaging of OSCC Xenografts Using a Dual-Axis Confocal Microscope

To show the feasibility of intravital tumor imaging at cellular resolution, excised subcutaneous FaDu xenografts, tongue, and muscle were imaged 90 minutes after PARPi-fl (75 nmol/167 μl PBS with 30% PEG300) or vehicle injection using a custom-built dual-axis confocal microscope. Illumination settings were optimal for BODIPY-FL imaging and settings (laser intensity and detector gains) were fixed for all tissues to ensure comparability (illumination intensity: 1.95-2.1 mW and photomultiplier gain setting: 0.656 V).

### Fluorescence Endoscopy

FaDu xenografts were imaged with a fluorescence endoscope that is available for both clinical and preclinical imaging (Cellvizio, Mauna Kea Technologies, Paris, France). It provides cellular to subcellular resolution and has a flexible confocal microprobe that enables versatile imaging. Here, after receiving a 90-minute post-injection of 150 nmol PARPi-fl (in 167 μl PBS with 30% PEG300) or vehicle (167 μl PBS with 30% PEG300), animals were sacrificed and skin was removed from subcutaneous FaDu tumors and thigh muscle (n=4 PARPi-fl, n=3 vehicle). The microprobe was slowly moved over the tumor, tongue, or muscle, while a real-time video was recorded using a 488 nm excitation beam. The videos were converted to grayscale and the intensity was measured in 10 frames per video using ImageJ 1.49e Software. Topical application of PARPi-fl

For topical application of PARPi-fl, mice with or without orthotopic tongue tumors (FaDu_{tdTomato}) were anaesthetized using ketamine (0.1 mg/g body weight) and tongues were exposed using forceps. For topical application, the tongues were dipped into a well of a 96-well plate filled with the respective incubation solution. The sequence of incubation was first 20 seconds in 1% acetic acid second 20 seconds PBS third 1 minute 5 μM PARPi-fl (30% PEG300/PBS) fourth 1 minute 1% acetic acid and fifth 10 seconds PBS. This was followed by cleaning of the tongue with an alcohol pad to remove residual unbound compound. The animals were imaged in the IVIS Spectrum before and after PARPi-fl application using the appropriate filter sets for detection of PARPi-fl and the tdTomato fluorescent protein. Spectral unmixing was used to separate the signals for tdTomato, PARPi-fl and autofluorescence. The tdTomato fluorescent protein allows in vivo localization of the tumor. For comparability, all images were scaled to the same maximum radiant efficiency. Imaging was repeated with sections of the excised tongues after cryofixation. Sections were fixated in 4% PFA, counterstained with Hoechst and imaged using a confocal microscope to localize PARPi-fl in the tissue. Adjacent sections were H&E stained for morphological evaluation.

### Statistical Analysis

Statistical analysis of preclinical data was performed using GraphPad Prism 6 and R 3.1 (www.r-project.org). Unless otherwise stated, data points represent mean values, and error bars represent standard deviations of biological replicates. P values were calculated using a Student's unpaired t-test, corrected for multiple comparisons by the Holm-Sidak method with an alpha of 0.05 as the cutoff for significance. For the clinical specimen, the distribution of the percent PARP1-positive area was separately estimated for normal and malignant tissues using kernel density estimation. The ability to use PARP1 expression to distinguish malignant tissues from adjacent normal tissue was characterized by a receiver operating characteristic (ROC) curve. The probability of a given tissue being malignant as a function of the PARP1-positive tissue area (in percent) was estimated by nonparametric binary regression using the method of local likelihood.

### Example 3: Optical Imaging of PARP1 in Response to Radiation in OSCC

PARPi-fl, in tissues both with and without prior DNA damage, was investigated as a probe for PARP1 imaging. It was shown that PARP1 expression in oral cancer is high, and that the uptake of PARPi-fl is selective, irrespective of whether cells were exposed to irradiation or not. It was also shown that PARPi-fl uptake increases in response to DNA damage, and that this increase is reflected in higher enzyme expression. These findings provide a framework for measuring exposure of cells to external beam radiation and for helping elucidate the effects of such treatments non-invasively in cancer subjects.

As described in the Background section, oral cancer is a type of malignant growth that more than 45,000 individuals will be diagnosed with in the United States in 2015 alone. Treatment options have improved over the last years, and the overall 5-year relative survival rates have increased from 52.7% in 1975 to 66.3% in 2007. This is in part due to the introduction of novel treatment options, one of which is intensity-modulated radiation therapy (IMRT). This type of radiation therapy allows the administration of ionizing radiation with varying intensities, effectively depositing DNA-damage events in a fairly defined region of the oral cavity. While IMRT is administered routinely, little is known about the spatial resolution of DNA damage on a case-by-case basis, and whether this damage can be visualized using injectable probes.

Although PARPi-fl was validated as an imaging agent for tumor tissue in the Examples described here, its use for tissues that underwent treatment has not been investigated. The Example provides: if PARPi-fl accumulates selectively in tumor nuclei, even after delivering a dose of radiation lethal to greater than 95% of a tumor cell population; if the marker is distributed and retained in tumor tissue, even after delivery of a therapeutic dose of radiation; and if PARP1 levels responds to ionizing radiation, and can this response be imaged using PARPi-fl.

The understanding of where, how, and to what extent radiation damage unfolds is critical to designing effective and optimized treatments regimens. The correlation between irradiation and DNA damage in oral cancer cells has been shown on the histological level, for example by measuring phosphorylated γH2AX foci formation. However, an injectable marker which can image such a response is to date an unmet clinical need.

It was shown herein that PARP1 is overexpressed in oral cancer. Using this model, it was determined in the present Examples that PARPi-fl is a selective marker in oral cancer cell lines, irrespective of whether they received ionizing radiation or not. The results described herein show that PARPi-fl uptake increases as a response to ionizing radiation within the first 48 hours. The results described herein also show that the elevated uptake correlates with higher PARP1 expression, and that uptake is selective not only in vitro, but also in vivo. Accordingly, PARP1 can serve as a marker of radiation-induced DNA damage.

### PARP1 Expression in Tissues

PARP1 antigen expression was assessed in mouse tongue, FaDu and Ca127 xenografts using IHC to determine their basic PARP1 expression before irradiation. The staining was done using the Discovery XT processor (Ventana Medical Systems, Tucson, Ariz.). Paraffin-embedded formalin fixed 3 μm sections were deparaffinized with EZPrep buffer, antigen retrieval was performed with CC1 buffer (both Ventana Medical Systems, Tucson, Ariz.) and sections were blocked for 30 min with Background Buster solution (Innovex, Richmond, Calif.). The anti-PARP1 rabbit polyclonal antibody (sc-7150, Santa Cruz Biotechnology, Santa Cruz, Calif.) was incubated for 5 h (0.2 μg/ml), followed by 1 hour incubation with biotinylated goat anti-rabbit IgG (PK6106, Vector Labs, Burlingame, Calif.) at a 1:200 dilution. For detection, a DAB detection kit (Ventana Medical Systems, Tucson, Ariz.) was used according the manufacturer instructions. Sections were counterstained with hematoxylin and coverslipped with Permount (Fisher Scientific, Pittsburgh, Pa., USA). Incubating with a rabbit IgG instead of the primary antibody controlled for non-specific binding of the secondary antibody. Adjacent sections were stained with hematoxylin and eosin for morphological evaluation of the tissue. The staining was performed at the Molecular Cytology Core Facility of MSK. For quantification of PARP1 protein distribution, thresholding was performed (MetaMorph® Software, Molecular Devices, Sunnyvale, Calif.) on brown (PARP1) and blue (tissue) areas of digitalized sections and the relative PARP1 positive area was calculated by dividing the brown area by the total tissue area. 10 field-of-views were analyzed per section.

### Cell Irradiation and Clonogenic Survival

Cells were irradiated with 0, 2, 4, 6, 8 and 10 Gy in 75 cm² culture flasks using a J.L. Shepherd Cesium irradiator (J.L. Shepherd, San Fernando, Calif.) at a dose rate of 174 cGy/min. Clonogenic survival was assessed. Briefly, after irradiation, cells were trypsinized, counted, and pre-defined numbers of viable cells were plated in 6-well plates in triplicate. In order to receive a sufficient colony count (e.g., from 50 and 100), two cell numbers were plated per irradiation dose (0 Gy: 200, 500; 126 2 Gy: 500, 1000; 6 Gy: 800, 3000; 8 Gy: 1600, 7000; 10Gy: 2500, 8000). Cells were cultured 10-14 days and then stained with 0.5% Crystal Violet (Sigma-Aldrich, St. Louis, Mo.) for 10 min at room temperature. Only colonies comprising at least 50 cells were counted, and a mean was calculated from the triplicate wells. The plating efficiency of each irradiation dose was calculated by dividing the number of counted colonies by the number of cells plated. The relative clonogenic survival was calculated by dividing the plating efficiency of a certain irradiation dose by the plating efficiency of untreated cells. Three independent experiments were carried out for each cell line.

### PARP1-fl Uptake of Cells

To determine the binding of PARPi-fl to cells, cells were plated in 8-well Chamber Slides (Lab-Tek Brand; Nalge Nunc International, Naperville, Ill.). After 24 hours, cells were treated with 0 or 10 Gy irradiation in a J.L. Shepherd Cesium irradiator (J.L. Shepherd, San Fernando, Calif.) at a dose rate of 174 cGy/min. 24 hours post irradiation, cells were incubated with a 1 μM solution of PARPi-fl for 20 min at 37° C., followed by two 5 min incubations in full medium and one wash in PBS. Subsequently, cells were fixed with 4% Paraformaldehyde, plastic chambers were removed and slides were mounted with Mowiol® mounting medium containing Hoechst 33342 for counterstaining of cell nuclei. Imaging was done using a Leica SP5 upright confocal microscope (Leica, Buffalo Grove, Ill.), equipped with appropriate lasers and emission filters. PARPi-fl was imaged using the FITC channel and 488 nm laser excitation.

### Effect of Cell Irradiation on PARPi-fl Uptake

The change in PARPi-fl uptake was quantified in FaDu and Ca127 cells after irradiation using Flow Cytometry. First, cells were irradiated with 0, 2, 4 and 10 Gy in 25 cm² culture flasks using the J.L. Shepherd Cesium irradiator (J.L. Shepherd, San Fernando, Calif.) at a dose rate of 174 cGy/min. At different time intervals post irradiation (6, 24 and 48 hours) PARPi-fl staining was initiated. Following a wash with PBS, cells were trypsinized, counted, and portions of 0.5×10⁶ cells of the single cell suspension were aliquoted into 1.5 ml Eppendorf tubes (Eppendorf, Hamburg, Germany). For each time point and irradiation dose, samples were either left unstained, were stained with PARPi-fl or olaparib/PARPi-fl. Co-incubation with a 10-fold excess of the non-fluorescent PARP1 inhibitor olaparib was carried out to control for binding specificity of PARPi-fl. For staining, cells were washed with 1 ml FACS buffer (1% BSA (w/v) in PBS). Then, 1 ml of the staining solution (FACS buffer only, 0.5 μM PARPi-fl in FACS buffer or 5 μM olaparib/0.5 μM PARPi-fl in FACS buffer) was added for 20 min at 37° C., followed by one 5 min wash in 1 ml FACS buffer. Next, cells were centrifuged, the supernatant was aspirated and cells were re-suspended in 0.5 ml FACS buffer and transferred to 5 ml round bottom flow cytometry tubes (BD Biosciences, Bedford, Mass.) through a 40 μm strainer to remove doublets and left on ice until measurement in the flow cytometer (LSR® II, BD Biosciences, Bedford, Mass.). For each measurement, 10,000 events were counted. Raw data were processed in FlowJo software in order to calculate the changes in PARPi-fl uptake after irradiation. Cell clumps and debris were eliminated using the corresponding gates (forward and side scatter) for the unstained cell population. The gates were applied to all stained samples. PARPi-fl fluorescence was imaged in the FITC channel against side scatter (area).

### In Vivo Irradiation

Bilateral FaDu xenografts were inoculated 15 days before irradiation on the left and right side of the lower back of female athymic nude mice (n≥3/group). Tumor volume was measured with a caliper every 3-5 days and calculated by the formula π/6×(length×width×height of the tumor). Tumors on the right side were irradiated with 10 Gy using an image-guided microirradiator (X-Rad 225 Cx, Presicion X-Ray, North Branford, Conn.). The irradiation area was centered on the tumor by using the built-in cone-beam CT for soft tissue imaging and a 2×2 cm collimator. X-Ray irradiation was delivered at a dose rate of 3.1306 Gy/min while animals were under 2% isoflurane anesthesia.

### PARP1 Expression Following Irradiation

At 24 hours and 48 hours after the irradiation, animals were sacrificed using carbon dioxide asphyxiation. Tumors were explanted, formalin-fixed and embedded in paraffin for immunofluorescent PARP1 staining. This was done following the protocol for IHC as described above, with the difference that detection was performed with Streptavidin-HRP D (from DABMap Kit, Ventana Medical Systems), followed by incubation with Tyramide Alexa Fluor 594 (T20935, Invitrogen, Carlsbad, Calif.), prepared according to the manufacturer's instructions. Sections were counterstained with 4′,6-diamidino-2-phenylindole (DAPI) for 10 min and coverslipped with Mowiol® mounting medium (Sigma-Aldrich, St. Louis, Mo.). Immunofluorescence staining allowed for evaluation of the intensity of the PARP1 signal in each nucleus in addition to the PARP1 positive area. In each section, 10 fields of view were analyzed (total area 3.64 mm²). For each tumor, three sections were analyzed. Per group (irradiated and non-irradiated) 4 tumors were analyzed. The PARP1 quantification was done on digitalized slides using an automated segmentation and quantification protocol generated with the software MetaMorph® (Molecular Devices, Sunnyvale, Calif.) using the three scanned channels. The PARP1 positive area was determined by thresholding the red fluorescent area and dividing it by the whole tissue area, which was determined based on autofluorescence. PARP1 intensity was determined by measuring the red fluorescence intensity in all nuclei, which were thresholded using DAPI staining. The measured fluorescence intensities were averaged over all nuclei in each field-of-view.

Synthesis of PARPi-fl with BODIPY-FL as the Fluorophore

Synthesis of an example optical imaging agent PARPi-fl with BODIPY-FL as the fluorophore was carried out in a manner analogous to that previously described above. Briefly, the green fluorescent dye BODIPY-FL NETS-ester (Invitrogen, Carlsbad, Calif.) was conjugated to 4-(4-fluoro-3-(piperazine-1-carbonyl)benzyl)phthalazin-1(2H)-one, followed by purification via preparative HPLC (Waters' XTerra C-18 5 μm column, 7 ml/min, 5% to 95% of acetonitrile in 15 min). PARPi-fl was obtained in 70-79% yield as a red solid in >97% purity. The identity of PARPi-fl was confirmed using ESI-MS (MS(+) m/z=663.4 [M+Na]+). For in vivo imaging studies, PBS (117 μl) was slowly added to an aliquot of PARPi-fl (50 μg, 75 nmol) in 50 μl of poly(ethylene glycol) (PEG300, Sigma-Aldrich, St. Louis, Mo.) to obtain a final injection volume of 167 μl.

### Imaging of PARPi-fl Uptake in Response to Irradiation

Cohorts of subcutaneous FaDu tumor bearing animals were injected intravenously with PARPi-fl (75 nmol/167 μl PBS with 30% PEG300) 24 hours and 48 hours post irradiation and 90 min before sacrifice by carbon dioxide asphyxiation. Irradiated and non-irradiated tumors as well as tongues were explanted and the fresh tissues imaged immediately in the epifluorescence system IVIS (PerkinElmer, Waltham, Mass.) using the standard filter set for GFP imaging. Autofluorescence was removed using spectral unmixing. The PARPi-fl signal was analyzed semiquantitatively by measuring the average radiant efficiency [p/s/cm²/sr]/[μW/cm²] in regions of interest (ROIs) that were placed on the tissue under white light guidance. Resulting numbers are normalized for the integration time, binning, f/216 stop, field of view, illumination intensity, and the ROI area, making measurements comparable among each other. After epifluorescence imaging, the freshly excised whole tumors were imaged microscopically. Tissues were placed on a cover slip with a freshly cut surface facing the cover slip and images were taken on an inverted laser scanning confocal microscope using 488 nm laser excitation (LSM 5-Live, Zeiss, Jena, Germany). PARPi-fl stained tumors were also compared to tumors that did not receive PARPi-fl injection to assess the extent of autofluorescence in the images. To confirm the specificity of the PARPi-fl stain to PARP1 protein, cryosections of the excised tumors were stained with an anti-PARP1 antibody. For this, 10 μm cryosections were fixed with 4% Paraformaldehyde, blocked for 30 min with 3% goat serum, stained overnight with the primary antibody (rabbit anti-PARP1, 1 μg/ml, sc-7150, Santa 226 Cruz), rabbit IgG (isotype control) or antibody dilution buffer (no primary control, PBS containing 1%(w/v) BSA and 0.3% TritonX-100). This was followed by secondary antibody staining (goat anti228 rabbit-AF680, 2 μg/ml, Invitrogen). Slides were mounted with Mowiol (Sigma-Aldrich) containing Hoechst 33342 for nuclear counterstaining.

### Statistical Analysis

Statistical analysis was performed using GraphPad Prism 6. Unless otherwise stated, data points represent mean values, and error bars represent standard deviations of biological replicates. P values were calculated using a Student's unpaired t-test, corrected for multiple comparisons by the Holm-Sidak method with an alpha of 0.05 or 0.01 as the cutoff for significance.

### Results

### PARP1 Expression in Tissues

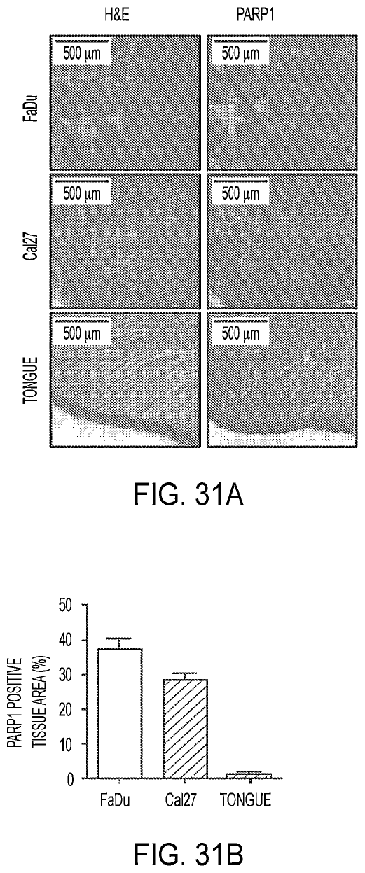

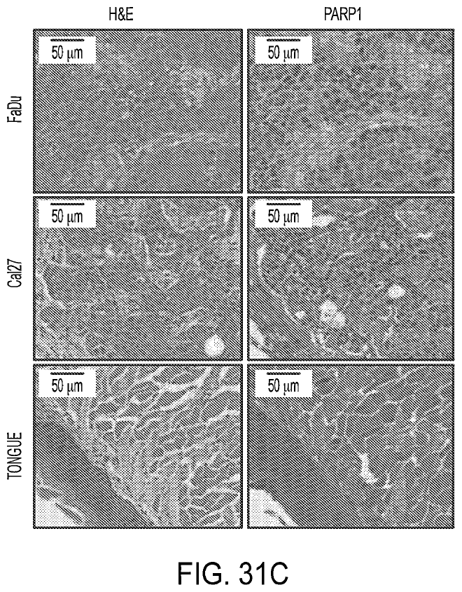

Strong nuclear expression of PARP1 was observed in FaDu and Ca127 tumor tissue, but not in mouse tongue tissue (FIG. 31A). PARP1 expression was quantified by measuring the percentage of tissue area that was stained by PARP1 (brown staining) compared to the whole tissue area (stained with hematoxylin; blue) using color thresholding. In FaDu tumors, 37.2±3.2% of the tissue was found to be positive for PARP1, and Ca127 tumors displayed PARP1 staining in 28.7±1.7% of the tissue. In contrast, tongue tissue (muscle and mucosa) had a 1.4±0.4% PARP1 positive area (FIG. 31B). Specificity of the staining became obvious in higher magnifications, where it was seen that only tumor cell nuclei displayed strong PARP1 staining, but not stromal tissue or muscle tissue (FIG. 31C).

Cell Survival and PARP1-fl Imaging after External Beam Irradiation

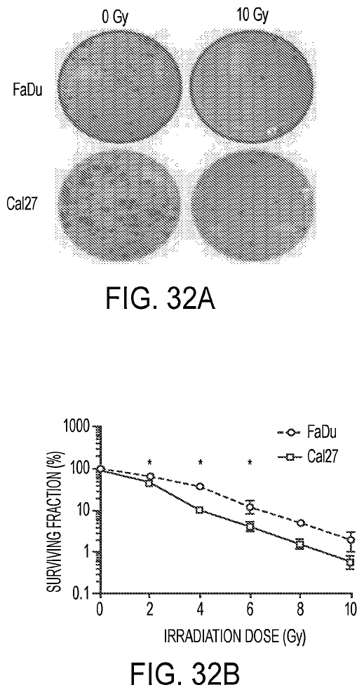

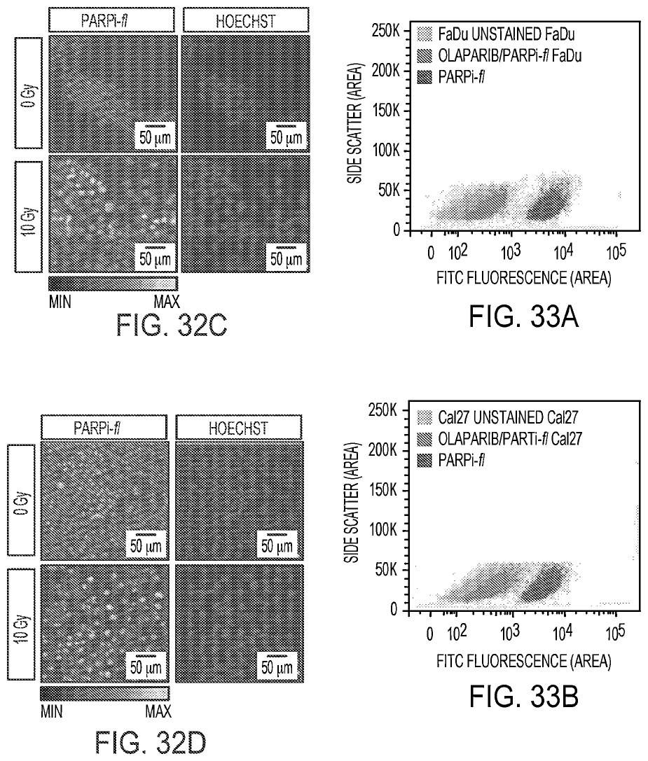

Before imaging the PARPi-fl uptake in response to external beam irradiation in HNSCC cell lines, the effect of irradiation was quantified on cell survival. Clonogenic assays revealed that cell survival decreased exponentially with increasing irradiation doses, as seen by reduction of the colony count (FIGS. 32B and 32C). At 2, 4 and 6 Gy, Ca127 cells were more sensitive to irradiation than FaDu cells, reflected by a significantly lower surviving fraction (p<0.05, FIG. 32B). The most pronounced difference was seen at 4 Gy, where 37±5% of FaDu cells, but only 10±2% of Ca127 cells maintained their clonogenicity. At 10 Gy, the surviving fraction was 2±1% for FaDu cells and 0.6±0.2% for 261 Ca127 cells, confirming the near-quantitative lethality of the external beam irradiation at this dose.

### Effect of Cell Irradiation on PARP1-fl Uptake

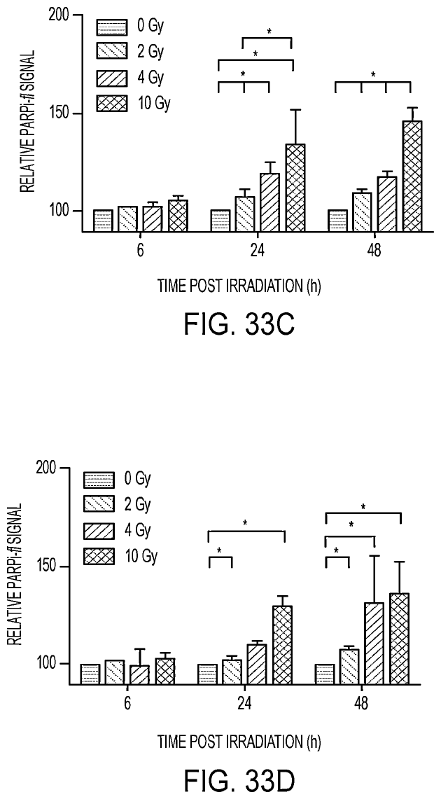

PARP1 expression of FaDu and Ca127 cells was imaged using the fluorescent PARP1 inhibitor PARPi-fl. A quantitative relation between PARP1 expression and PARPi-fl binding was described above. PARPi-fl accumulated in the nuclei of FaDu and Ca127 cells, irrespective of the fact whether cells were irradiated with 10 Gy or not (FIGS. 32C and 32D). To quantify the impact of PARP1 expression and PARPi-fl uptake, in vitro assays were performed at different time points and a set of irradiation doses. In non-irradiated cells, both cell lines showed a strong uptake of PARPi-fl (measured in the FITC channel), which separated the PARPi-fl incubated population from an unstained cell population (FIGS. 33A and 33B). The imaging agent uptake was almost completely suppressed when the non-fluorescent PARP1 inhibitor olaparib was co-incubated with PARPi-fl (50-fold excess). This effect showed specificity of PARPi-fl, because olaparib and PARPi-fl, which are both derived from the same scaffold, compete for the ADP binding site on PARP1. Cells were irradiated with 2, 4 and 10 Gy and PARPi-fl uptake was quantified 6, 24 and 48 hours after the irradiation. At each time point, the mean fluorescence intensity was compared to non-irradiated cells (0 Gy), and the relative PARPi-fl signal was calculated. In FaDu cells, no statistically significant differences between cell populations receiving different radiation doses were observed 6 hours post treatment. At 24 and 48 hours, however, all irradiation doses led to an increase in PARPi-fl uptake compared to the non-irradiated cells (FIG. 33C). The strongest increase of PARPi-fl uptake was observed 48 hours after 10 Gy irradiation, where the PARPi-fl signal was 145.4±7.3% compared to non-irradiated cells. Comparable results were observed in Ca127 cells. At 6 hours post irradiation, the PARPi-fl uptake was comparable for all cell populations. At 24 and 48 hours post irradiation, however, the PARPi-fl signal gradually increased with increasing irradiation doses (FIG. 33D). The most pronounced effect was again observed at 10 Gy irradiation and 48 hours post treatment (136.0±16.6% compared to non-irradiation cells). When compared to FaDu cells, 4 Gy irradiation caused a stronger increase in PARPi-fl uptake 48 hours post irradiation.

### Effect of In Vivo Irradiation on PARP1 Expression and PARPi-fl Uptake

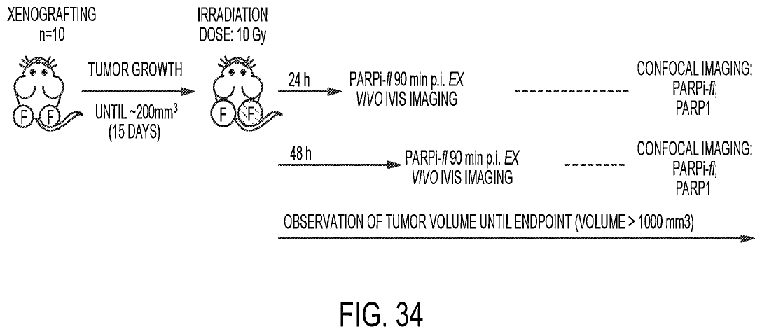

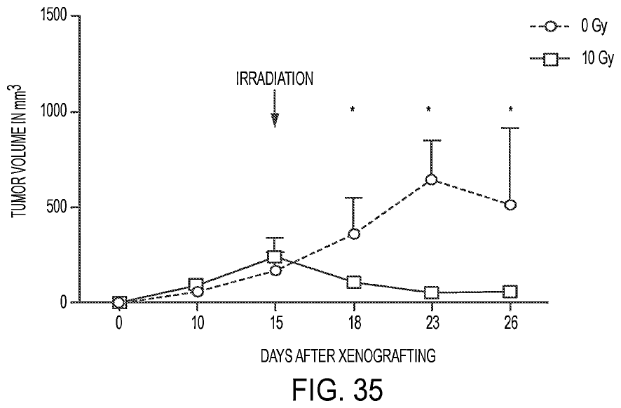

The effect of 10 Gy irradiation on PARP1 expression and PARPi-fl uptake was assessed in bilateral FaDu tumor bearing nude mice, where the tumor on the right flank was exposed to 10 Gy using an image-guided microirradiator on day 15 after tumor inoculation (FIG. 34). The impact of irradiation was also monitored by looking at the tumor volume for up to 26 days after the tumor 311 inoculation (FIG. 35). In the non-irradiated tumors, the volume gradually increased from 171±92 mm³ on day 15 to 512±403 mm³ until the end of the observation. Irradiation, on the other hand, led to an immediate reversal of the tumor growth curve and a decrease of mean tumor volume from 230±327 mm³ on day 15 to 54±14 mm³ on day 26.

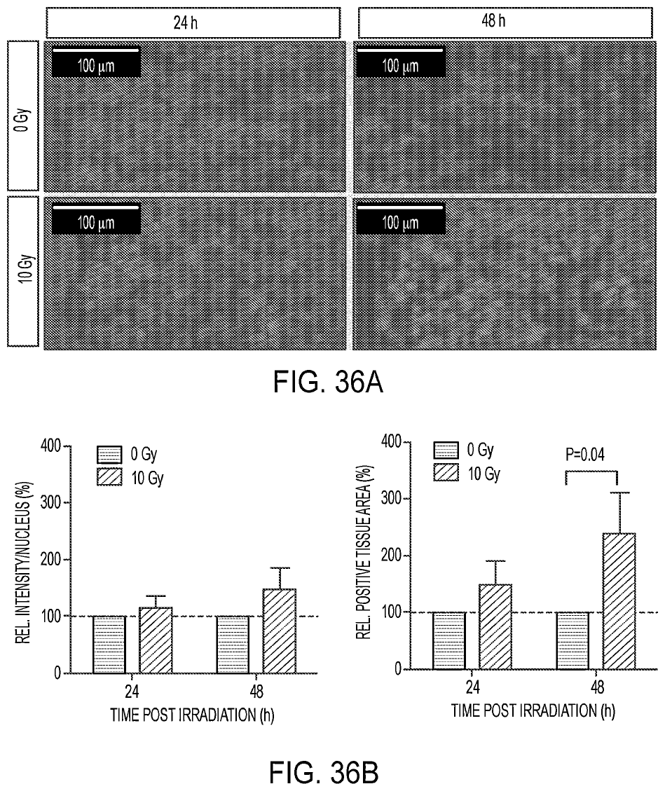

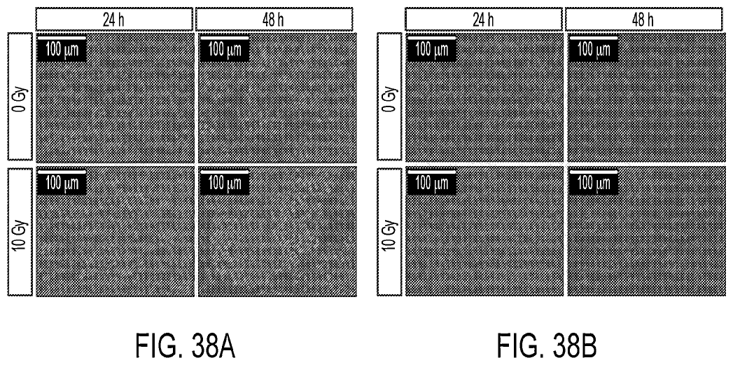

Tumor sections of irradiated and non-irradiated tumors were stained for PARP1 using Immunofluorescence staining at 24 and 48 hours post irradiation (FIG. 36A). Analogous to the in vitro experiments, increased expression of PARP1 was observed in tissues that had been exposed to 10 Gy leading to higher PARP1 expression levels in individual nuclei (FIG. 36B), with relative intensities of 114±21% (24 hours post irradiation) and 147±38% (48 hours post irradiation) compared to non-irradiated (0 Gy) tumor tissues. The PARP1 positive area was increased to a much higher degree than PARP1 expression, an effect that might be amplified by the influx of immune cells, which can themselves produce high levels of PARP1. After 24 hours, the PARP1 positive area was increased to 150±42%, compared to non-irradiated tumors. After 48 hours, the PARP1 positive area more than doubled (239±73%, p=0.04) in irradiated vs. non-irradiated tumors (FIG. 36B). Irradiation led to an increase in both PARP1 expression and density in FaDu tumors. The specificity of the staining was confirmed at all observed time points and irradiation doses by using a rabbit IgG as primary antibody, which did not lead to nuclear or non-nuclear staining (FIG. 38A and FIG. 38B).

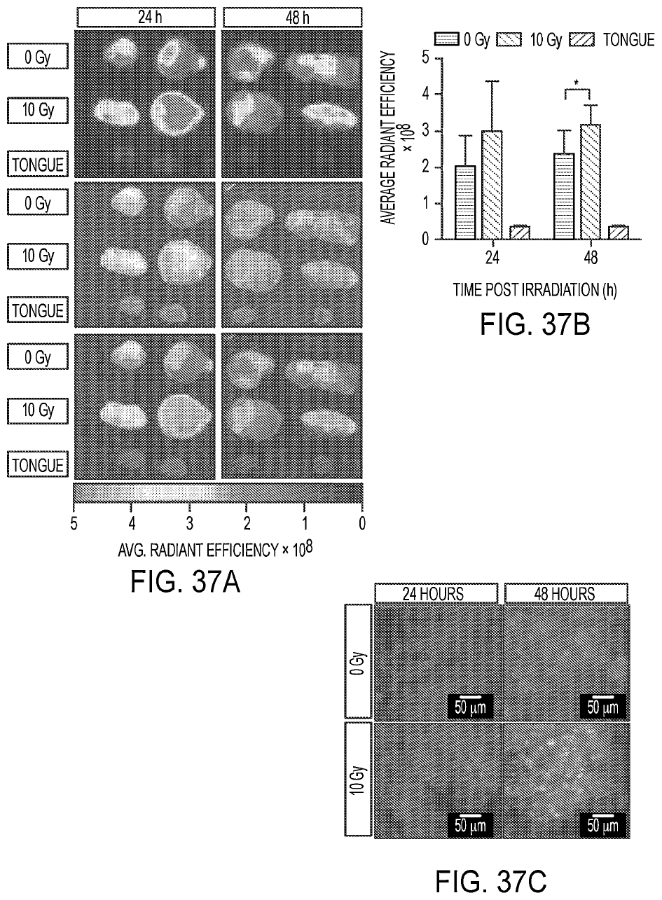

Determining the uptake of PARPi-fl macroscopically in freshly excised tumor tissues at 24 and 48 hours after irradiation, it was found that an increased uptake of PARPi-fl, mirroring the pattern of PARP1 protein expression (FIGS. 37A-37C). Specifically, epifluorescence imaging of excised FaDu tumors revealed a statistically significant increase of the average radiant efficiency 48 hours after external beam irradiation (radiance levels were 2.3 0.7×10⁸ and 3.2±0.6×10⁸ for non-irradiated and irradiated tumors 48 hours after treatment, FIGS. 37A and 37B; p=0.047), while uptake of PARPi-fl into the normal tongue was negligible (0.3±0.05×10⁸). This trend was also observed on the microscopic level, using confocal microscopy (FIG. 37C). Here, too, the strongest nuclear fluorescence was observed in tumors at 48 hours after 10 Gy irradiation.

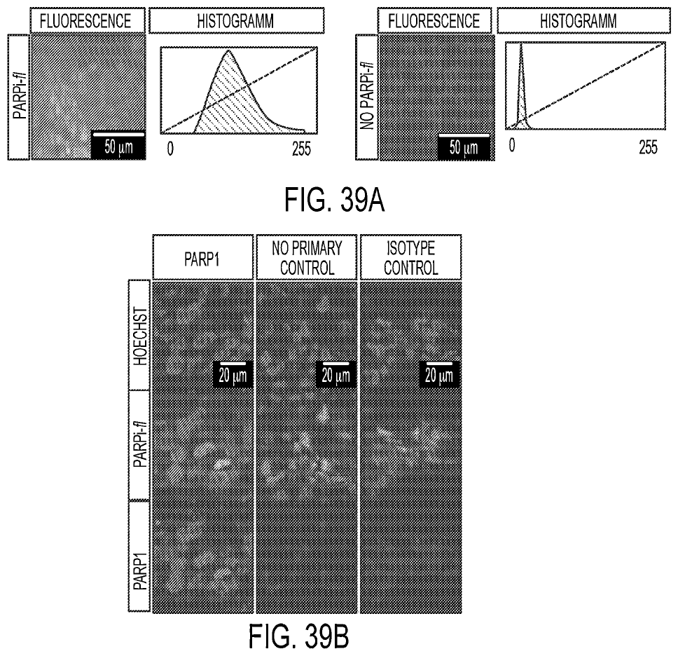

To assess the influence of autofluorescence on the signal in the green fluorescence channel, a FaDu tumor, which had not received PARPi-fl, was also observed. Here, a low autofluorescence signal with a very narrow histogram, as opposed to the PARPi-fl containing tumor, which displayed a right shift of the histogram curve and much broader distribution of the fluorescent intensities, was observed (FIG. 39A). To ensure that the PARPi-fl staining is specific to the PARP1 protein, cryosections were co-stained with PARP1 antibody and correlated the PARPi-fl and PARP1 signal. To exclude the possibility of an increase of nonspecific staining after irradiation, a tumor was chosen that was irradiated with 10 Gy 48 hours prior to PARPi-fl staining It was found that a specific localization of the PARPi-fl signal in nuclei (Hoechst staining) expressed PARP1 (FIG. 39B).

### DISCUSSION

Visualizing and quantifying the amount of external beam radiation delivered to a particular tissue compartment is a major challenge in radiation oncology research. Current methods used for determining the amount of radiation deposited in a given compartment largely rely on theoretical models and externally measured beam intensities. It has been recognized that such models have experienced considerable advances in past decades, but are also becoming prone to error with increasing complexity.

The Examples herein established a molecular imaging approach using rapid PARP1 targeted fluorescence imaging to yield a reproducible measure of the effects of external beam radiation to oral cancer tissue. The data shows that PARP1 is a robust biomarker, and that the agent accumulates selectively in OSCC cells both in vitro and in vivo, with and without previous irradiation treatment. It was also shown that PARP1 indeed responded to ionizing radiation, and that this change can be seen with PARPi-fl for both in vitro and in vivo experiments.

The disclosed imaging approach is based on the strongly elevated PARP1 expression in cancer tissue compared to its healthy surrounding host tissue. Specifically, the described xenograft mouse models showed that PARP1 expression was, with levels of 37.2±3.2% and 28.7±1.7% (for FaDu and Cal 27, respectively), 26-fold and 21-fold higher in tumor tissue than tongue tissue (1.4±0.4%, FIGS. 31A-31C). When exposed to up to 10 Gy of external beam radiation, the clonogenic potential of both FaDu and Ca127 was heavily impacted. It was reduced to 2.0±1.0% and 0.6±0.2% for FaDu and Ca127, respectively. In contrast, at short time points after irradiation (between 6 and 48 hours), cells maintained their viability and PARPi-fl staining did not show qualitative differences from non-irradiated cells. Furthermore, specificity of the PARPi-fl uptake was not affected, as shown by blocking studies.

While no changes in PARPi-fl uptake 6 hours after radiation exposure were seen, median PARPi-fl uptake changed at 24 hours, and particularly at 48 hours after an irradiation event. Changes were seen for as little as 2 Gy, and were more pronounced with increasing dose (FIGS. 33A-33D). The same effect was observed in vivo, where PARP1 expression of tumors that were treated with radiation were compared to those that did not receive treatment. Specifically, mice with bilateral FaDu tumors, one of which was treated with 10 Gy of radiation, were used (FIG. 34). In concordance with in vitro experiments described herein, the radiation treatment had a major effect on cell viability, resulting in a tumor volume in the treated group that was 10 times lower than in the non-irradiated tumors (FIG. 35). On histological tumor sections, the treatment resulted in an increase of PARP1 expression similar to that seen using flow cytometry in vitro (1.5±0.4 fold increase in fluorescence/nucleus in treated versus untreated FaDu xenografts 48 hours post irradiation). Not only the expression, but also the density of PARP1 expressing cells increased in tumors at 48 hours after irradiation. Without wishing to be bound to any theory, this can be due to elevated PARP1 expression in a subset of tumor cells that were expressing low levels of PARP1 before treatment, but can also be a response of the tumor to the irradiation, and resulting immune cell recruitment. The increase in PARP1 expression in individual nuclei, paired with higher nuclear densities after irradiation, can also be detected using ex vivo whole tumor imaging after PARPi-fl administration (FIGS. 37A-37C). It was found that the median radiant efficiency of tumors that were irradiated was 3.2±0.6×10⁸ at 48 hours post treatment. Tumors without irradiation had a radiant efficiency of 2.3±0.7×10⁸ at 48 hours post treatment. This increase is in the same range as the increase in PARP1 protein expression, suggesting that PARP1 expression levels can be measured on the whole tumor level using PARPi-fl imaging. The selectivity of PARP1 staining in vivo was confirmed by confocal imaging, which showed that the overwhelming amount of imaging agent accumulated in cell nuclei, irrespective of whether those were treated with external beam radiation or not. Confocal imaging also showed that 24 and 48 hours after 10 Gy irradiation, staining with PARPi-fl was more intense in individual nuclei, but also more diffuse and diverse (FIG. 37C). The irradiation treatment can also affect the tumor microenvironment, resulting in compromised tumor tissue architecture, dead cells, disrupted perfusion, and the subsequent effects on tumor penetration and clearance of PARPi-fl in the tumor.

The increased expression of PARP1 post irradiation also provides for a combination of radiation therapy with PARP1 inhibitor therapy to mediate synthetic lethality to tumor tissue.

Accordingly, it was shown that PARP1 expression increases in response to external beam radiation, and that this increase can be observed in cell culture and on the tissue level. Further, the fluorescent imaging agent PARPi-fl is able to accumulate in irradiated cell nuclei of tumor tissues. Such accumulation indicates that PARP1 targeted imaging agents can be used to delineate tissues exposed to radiation. For examples, PARP1 targeted imaging agents can be used to elucidate the effects of changing perfusion, cell density and other architectural changes inside the tumor.

In other applications, PARP1 imaging can be applied to other modalities, for example whole body PET imaging, using ¹⁸F labeled or dual labeled (e.g., ¹⁸F and Bodipy-FL). PARP Inhibitors can be critical to enable clinical PARP1 imaging and a quantitative relationship between PARP1 expression in whole body PET imaging post irradiation and therapy outcome can be determined based on the disclosure herein.

### Methods

### Cell Culture

Experiments were carried out using two human OSCC cell lines. FaDu (hypopharyngeal SCC; ATCC, Manassas, Va.) were maintained in MEM medium and Ca127 cells (tongue SCC; ATCC, Manassas, Va.) were maintained in D-MEM medium, both containing 10% (v/v) FBS and 1% Penicillin/Streptavidin. Cells were grown in monolayer culture at 37° C. in a 5% CO2 humidified atmosphere and passaged at 70-80% confluency.

### Animal Models

Female athymic nude (NCr-Foxnlnu, Taconic, Hudson, N.Y.) were housed under standard conditions with water and food ad libitum. Animals were anesthetized with 2% isoflurane throughout tumor implantation, irradiation and imaging. To implement subcutaneous human OSCC tumors, 2×10⁶ FaDu or Ca127 cells were dispensed in 50 μl medium, and 50 μl Matrigel™ (BD Biosciences, Bedford, Mass.) was added before injection on the lower back of the animals. For irradiation experiments, bilateral FaDu xenografts were used. Experiments were started 15 days after xenografting, when tumors had reached 100-150 mm³ volume. All animal experiments were done in accordance with institutional guidelines and approved by the IACUC of MSK and followed NIH guidelines for animal welfare. Animals were sacrificed before the experimental endpoint if tumors reached a volume of more than 1000 mm³, or animals displayed severe signs of distress such as rapid weight loss, crouching and impaired movement.

### Example 4: Optical Imaging of PARP1 in Human Subjects

In certain embodiments, for PARPi-fl imaging, patients can first gargle a solution of PARPi-fl for 1 min, then spit out this solution and gargle with a cleaning solution (e.g., the solvent used for PARPi-fl) for 1 min. Then fluorescence imaging of the oral cavity and pharynx can be performed with an endoscope for approximately 10-30 min. The intensity and extent of the fluorescence signal can be recorded for the tumor and adjacent normal mucosa.

In certain embodiments, PARPi-fl is stored as lyophilized powder and is reconstituted within 1 h of application. The final concentration of the PARPi-fl can range between 100-1000 nM. The solvent can be 15% PEG300/15% sorbitol in 70% water.

In certain embodiments, fluorescence imaging can be performed with a multispectral fluorescence camera. The camera can be mounted on a short rigid endoscope that is routinely used for the clinical examination of OSCC patients. The camera comprises a charged coupled digital (EM-CCD) camera for sensitive fluorescence detection and two separate cameras for detection intrinsic fluorescence and color. This system allows to correct the fluorescence images for the autofluorescence of the mucosa and to overlay the corrected fluorescence images in realtime on the color (photographic/video) image. The system attains a variable field of view (FOV) of 15 cm×15 cm to 3 cm×3 cm with a corresponding resolution from 150 μm to 30 μm.

The intensity of the fluorescence signal in the tumor region and adjacent normal mucosa will be determined and documented on digital images. On the fluorescence images also the area considered as suspicious for tumor will be determined and compared in a descriptive way with the area considered as tumor on the non-fluorescent, color image of the tumor region.

In Phase I, patients in a first cohort can receive escalating concentrations of PARPi-fl (e.g., 100, 250, 500 and 1000 nM). If there are no dose limiting toxicities (e.g., local irritation, pain, systemic effects) in the three patients then the next cohort of three patients can receive next escalated concentrations of PARPi-fl. If there is at least one toxicity in the cohort of three patients then the concentration below this dose level can be recommended for Phase II concentration of PARPi-fl. This design follows the popular 3+3 design for finding the maximum patients if one toxicity is seen in the first set of three patients at a given dose level.

The reasons for this is two-fold: it is not anticipated that any toxicity at any of the concentration levels used and acceptable levels of toxicity for an imaging agent is much lower than that of a therapeutic. Despite the anticipation of no toxicity, the choice of the design (escalating levels with toxicity as the primary endpoint) reflected the general concerns of a first-in-man study. Furthermore, it is possible that at a certain level of concentration unspecific binding of PARPi-fl can decrease image contrast. Therefore, the ratio of tumor fluorescence to fluorescence in surrounding mucosa at each dose level can be measured. If the fluorescence ratio decreases from one level to the next higher one by more than 2.5 standard deviations of the lower level, the dose escalation can be stopped, and the level that produced higher contrast for the Phase II study can be used.

In phase II, using the concentration established in phase I, 18 additional patients can undergo the same imaging procedure as described above within 4 hours prior to planed tumor resection. Images can be recorded as for phase I and tumor-to-normal ratios calculated for the fluorescence signal. Areas on the image that have a fluorescence signal that is at least 2-times higher than in the contralateral mucosa can be marked as tumor.

Gold standard can be obtained through the pathologic analysis of the surgical specimen and each area marked as tumor by fluorescence imaging will have the corresponding gold standard obtained. Since imaging is performed within hours of surgery, it is not expected that patients will need to be replaced. In addition malignant areas in the surgical sample that were missed on the images can be found. Sensitivity can be estimated in the following way: number of areas identified as malignant by imaging divided by the total number of malignant lesions by gold standard. Confidence intervals for this will be estimated taking into account the multiple observations from each patient. With, for example, 18 patients and an average of two lesions per patient the confidence interval can be estimated to within +/−14% assuming a true sensitivity of 80% and within-patient correlation of 0.1 (in the absence of previous clinical studies, this number is based on the data from other imaging modalities).

Correlation between PARP1 expression and fluorescence signal can be estimated by rank methods using the fluorescence signal intensity, and the intensity of PARP1 staining on the areas where the intensity was obtained. Delineation of tumor infiltration by PARPi-fl imaging can be assessed by studying the fresh frozen samples under a fluorescence microscope, and the fluorescence from PARPi-fl can be compared with HE staining of an adjacent section (as described herein and, for example, FIGS. 22A-22C). Results can be summarized as binary (e.g., infiltration under microscope yes/no, HE staining yes/no), presented as % concordant and compared with a McNemar test.

Table 3 shows exemplary PARP1 inhibitors that are binding to the same location (e.g. ABT-888, Abbott; AG014699, Pfizer; AZD2281, Astra-Zeneca; BMN-673, Biomarin; MK-4827, Merck). PARP1 imaging allows physicians to stratify patients in their appropriate treatment groups, enabling clinical decision making processes based on PARP1 levels.

TABLE 3

| PARP1 | | |
|---|---|---|
| inhibitor | IUPAC name | Chemical structure |
| AZD2281 | 4-[(3-[(4-cyclopropyl- carbonyl)piperazin-4- yl]carbonyl)-4- fluorophenyl]methyl (2H)phthalazin-1-one | 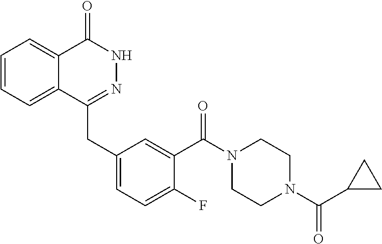 [SMILES: O=C(c1cc(Cc2n[nH]c(=O)c3ccccc23)ccc1F)N1CCN(C(=O)C2CC2)CC1] |
| AG014699 (rucaparib) | 8-Fluoro-2-{4-[(methyl- amino)methyl]phenyl}- 1,3,4,5-tetrahydro-6H- azepino[5,4,3-cd]indol- 6-one | 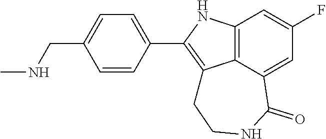 [SMILES: CNCc1ccc(-c2[nH]c3cc(F)cc4c3c2CCNC4=O)cc1] |
| ABT888 | 2-((R)-2-Methylpyrrolidin- | |
| (veliparib) | 2-yl)-1H-benzimidazole- | |
| | 4-carboxamide | |
| BSI201 | 4-iodo-3-nitrobenzamide | |
| (iniparib) | | |
| DR2313 | 1,5,7,8-Tetrahydro-2-methyl- 4H-thiopyrano[4,3-d] pyrimidin-4-one | 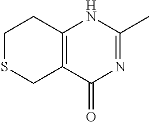 |
| FR 247304 | 5-chloro-2-[3-(4-phenyl-3,6- dihydro-1(2H)-pyridinyl) propyl]-4(3H)-quinazolinone | 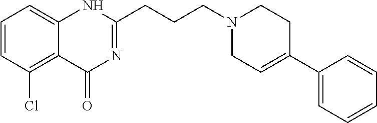 [SMILES: O=c1nc(CCCN2CC=C(c3ccccc3)CC2)[nH]c2cccc(Cl)c12] |
| GPI15427 | 10-(4-methyl-piperazin-1- ylmethyl)-2H-7-oxa-1,2- diazabenzo[de]anthracen- 3-one | 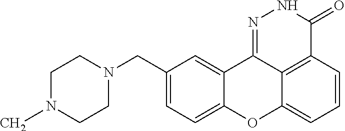 [SMILES: CN1CCN(Cc2ccc3c(c2)-c2n[nH]c(=O)c4cccc(c24)O3)CC1] |
| GPI16539 | 2-(4-methyl-piperazin-1-yl)- 5H-benzo[c][1,5]naphthyridin- 6-one | 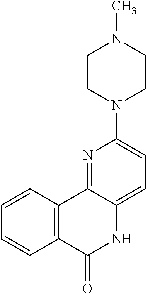 [SMILES: CN1CCN(c2ccc3[nH]c(=O)c4ccccc4c3n2)CC1] |
| MK-4827 | 2H-Indazole-7-carboxamide, 2-[4-(3S)-3-piperidinyl- phenyl]-, hydrochloride (1:1) | 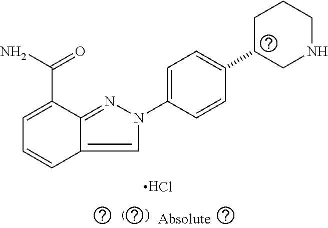 |
| NU1025 | 8-Hydroxy-2-methylquina- zoline-4-one | 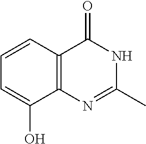 |
| NU1064 | 1H-Benzimidazole-7-carboxa- mide, 2-methyl- | 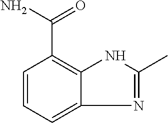 |
| NU1085 | 2-(4-hydroxyphenyl) benzimidazole- 4-carboxamide | 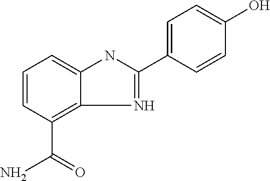 [SMILES: NC(=O)c1cccc2[nH]c(-c3ccc(O)cc3)nc12] |
| PD128763 | Adenosine, 5′-deoxy-5′-[4- [2-[(2,3-dihydro-1-oxo-1H- isoindol-4-yl)amino]-2- oxoethyl]-1-piperazinyl]- 5′-oxo- | 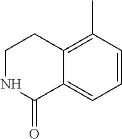 |
| PARP Inhibitor Π (INH2BP) | 5-iodo-6-amino-1,2- benzopyrone | 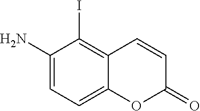 [SMILES: Nc1ccc2oc(=O)ccc2c1I] |
| PARP Inhibitor m (DPQ) | 3,4-Dihydro-5-[4-(1- piperidinyl)butoxyl]- 1(2H)-isoquinolinone | 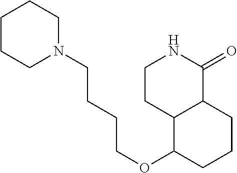 [SMILES: O=C1NCCC2C(OCCCCN3CCCCC3)CCCC12] |
| PARP Inhibitor VHI (PJ34) | N-(6-Oxo-5,6- dihydrophenanthridin- 2-yl)-(N,N-dimethyl- amino)acetamide | 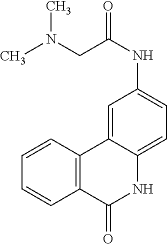 [SMILES: CN(C)CC(=O)Nc1ccc2[nH]c(=O)c3ccccc3c2c1] |
| PARP Inhibitor IX (EB-47) | H-isoindol-4-yl)amino]-2- oxoethyl]-1-piperazinyl]- 5′-oxo- | 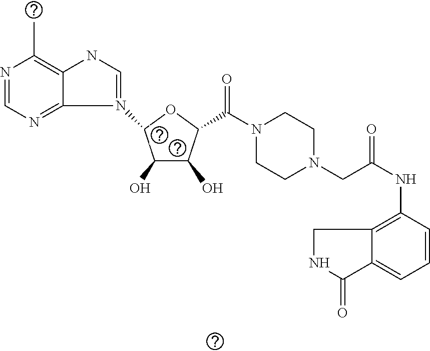 |
| TIQ-A | Thieno[2,3-c]isoquinolin- 5-one | 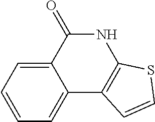 [SMILES: O=c1[nH]c2sccc2c2ccccc12] |

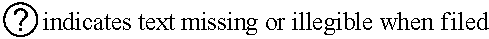

1.-20. (canceled)

21. A method for intraoperative detection of a tumor, tumor margin, and/or residual tumor tissue during glioblastoma removal surgery, the method comprising the steps of:
administering a liquid composition onto and/or into tissue in the brain of a subject, wherein the liquid composition comprises 100-1,000 nM of a PARP1 inhibitor conjugated to a fluorophore, and wherein the liquid composition is in contact with the tissue for about 1 minute;
flushing the tissue to reduce or remove unbound components of the liquid composition while leaving the bound components of the liquid composition; and
detecting fluorescence from the fluorophore after the administering and the flushing steps, thereby identifying the tumor, tumor margin, and/or residual tumor tissue.

22. The method of claim 1, wherein the fluorophore comprises a boron-dipyrromethane.

23. The method of claim 1, wherein the PARP1 inhibitor conjugated to a fluorophore is: [chemical structure]
